(19) United States
(12) Patent Application Publication
(10) Pub. No.: US 2026/0166179 A1
(43) Pub. Date: Jun. 18, 2026

(54) MUSCLE-SPECIFIC BASE EDITORS FOR CORRECTION OF MUTATIONS CAUSING DILATED CARDIOMYOPATHY

(71) Applicants: The Board of Trustees of the Leland Stanford Junior University, Stanford, CA (US); European Molecular Biology Laboratory, Heidelberg (DE)

(72) Inventors: Lars M. Steinmetz, Stanford, CA (US); Markus Grosch, Stanford, CA (US)

(73) Assignees: The Board of Trustees of the Leland Stanford Junior University, Stanford, CA (US); European Molecular Biology Laboratory, Heidelberg (DE)

(21) Appl. No.: 19/124,259

(22) PCT Filed: Nov. 8, 2023
(86) PCT No.: PCT/US2023/079074

§ 371 Date: Apr. 24, 2025

Related U.S. Application Data
(60) Provisional application No. 63/423,716, filed on Nov. 8, 2022.

(51) Int. Cl. A61K 48/00; A61K 9/00; A61P 9/00; C12N 5/074; C12N 5/077; C12N 9/22; C12N 9/78; C12N 15/11; C12N 15/86; C12N 15/87

(52) U.S. Cl. CPC A61K 48/0058; A61K 9/0019; A61K 48/0075; A61P 9/00; C12N 5/0657; C12N 5/0696; C12N 9/226; C12N 9/78; C12N 15/111; C12N 15/86; C12N 15/87; C07K 2319/80; C12N 2310/20; C12N 2506/45; C12N 2510/00; C12N 2750/14143; C12Y 305/04002; C12Y 305/04005

(57) ABSTRACT

Provided herein are compositions and methods for treating or preventing dilated cardiomyopathy (DCM) in a subject in need thereof, e.g., through correcting one or more point mutations at the RBM20 locus using CRISPR-associated base editing.

### CROSS-REFERENCES TO RELATED APPLICATIONS

This application claims priority to U.S. Provisional Application No. 63/423,716, filed Nov. 8, 2022, the disclosure of which is herein incorporated by reference in its entirety for all purposes.

### BACKGROUND

Dilated cardiomyopathy (DCM) is a heterogeneous disease with multiple causes and a non-specific phenotype that ultimately leads to the dilation of the left ventricle and systolic dysfunction. DCM is the second highest cause of heart failure with an estimated prevalence rate of 1:250. Current therapies for DCM include those broadly used for the management of heart failure, however these therapies, at best, can only slow DCM progression. Therefore, developing a strategy that maintains or even restores normal heart function would be a breakthrough for patients with dire treatment options.

Besides environmental causes of DCM (e.g., viral infection, toxins, inflammation), approximately 30% of all cases are due to inherited mutations of several structural components of the heart. A curation of DCM-associated genes identified 19 genes that exhibit a strong connection to DCM based on genetic and experimental evidence, and the RBM20 gene is one of them. The RBM20 gene encodes for a cardiac splice factor which regulates alternative splicing of genes critical for the function of cardiomyocytes. About 2-6% of patients with a highly penetrant and aggressive form of familial DCM have RBM20 mutations.

### BRIEF SUMMARY

The present disclosure provides methods and compositions for treating or preventing dilated cardiomyopathy (DCM) in a subject in need thereof, e.g., through correcting one or more point mutations at the RBM20 locus of cells taken from a subject, and subsequently reintroducing the genetically modified cells back into the subject. As a non-limiting example, the present methods and compositions involve CRISPR-associated base editing combined with target-specific viral delivery using a myotropic adeno-associated virus (AAVMYO) to achieve gene repair for treatment and prevention of DCM and other hereditary cardiac diseases.

Accordingly, in one aspect, the present disclosure provides a method for correcting a point mutation at the RBM20 locus in a cell, the method comprising: introducing into the cell (i) a single guide RNA (sgRNA) targeting a sequence comprising the point mutation and (ii) a base editor (BE), wherein the sgRNA binds to the base editor and directs it to the target sequence, whereupon the base editor corrects the point mutation at the RBM20 locus in the cell. In some embodiments, the method further comprises isolating the cell from the subject prior to the introducing of the sgRNA and the BE.

The method disclosed herein can correct any point mutations at the RBM20 locus or other loci associated with DCM in the cell. In some embodiments, the point mutation at the RBM20 locus comprises a substitution at an amino acid position comprising 83, 455, 535, 633, 634, 635, 636, 637, 638, 703, 716, 783, 831, 888, 913, 914, 1031, 1081, 1182, 1206, or a combination thereof; and wherein the substitutions and the positions are determined with reference to SEQ ID NO: 10. In some embodiments, the point mutation at the RBM20 locus comprises a substitution at an amino acid position comprising 633, 634, 635, 636, 637, 638, or a combination thereof. In some embodiments, the point mutation at the RBM20 locus comprises a substitution at amino acid position 633, 634, or a combination thereof. In particular embodiments, the substitution comprises P633L, R634Q, or a combination thereof.

Any sgRNA targeting a sequence comprising the point mutation of interest can be used in the claimed method. In some embodiments, the sgRNA comprises a sequence having about 80% or greater identity to any one of SEQ ID NOs: 1-9. In particular embodiments, the sgRNA comprises a sequence of any one of SEQ ID NOs: 1-9.

In some embodiments, the BE is an adenine base editor (ABE) or a cytidine base editor (CBE). In some embodiments, the BE comprises an RNA-guided catalytically impaired nuclease fused to a nucleobase deaminase enzyme. In some embodiments, the RNA-guided catalytically impaired nuclease is a dead Cas9 (dCas9), dCas12, or Cas9 nickase (Cas9n), or a derivative thereof. In some embodiments, the RNA-guided catalytically impaired nuclease is an engineered Cas9n. In some embodiments, the engineered Cas9n is Cas9n-NRNH, Cas9n-NRTH, Cas9n-NRCH, CP-1041, or SpRY.

In some embodiments, the nucleobase deaminase enzyme is a single-stranded DNA (ssDNA)-specific deaminase enzyme. In some embodiments, the deaminase enzyme is an adenine deaminase or a cytidine deaminase. In some embodiments, the BE is BE1, BE2, BE3, BE4, ABE6.3, ABE7.8, ABE7.9, ABE7.10, BE4max, AncBE4max, ABEmax, ABE8e, ABE-SpRY, CBE-SpRY, ABE-CP-1041, and CBE-CP-1041.

In some embodiments, the sgRNA and the BE are introduced into the cell in one of more expression cassettes. In some embodiments, the BE is present in one expression cassette. In some embodiments, the BE is present in two expression cassettes, wherein an active BE is packaged in the cell through intein-mediated trans-splicing. In some embodiments, the expression cassette comprises a promoter. In some embodiments, the promoter is a CAG promoter. In some embodiments, the promoter is a human cardiac troponin T (hTNNT2) promoter.

In some embodiments, the sgRNA and the BE are introduced into the cell using a recombinant adeno-associated virus (rAAV) vector. In some embodiments, the rAAV vector is an AAVMYO vector. In some embodiments, the sgRNA and the BE are introduced into the cell as a ribonucleoprotein (RNP). In some embodiments, the RNP is introduced into the cell by electroporation.

In some embodiments, the cell is an induced pluripotent stem cell (iPSC) or a cardiomyocyte derived from the iPSC (CM-iPSC).

In another aspect, the present disclosure provides a method for treating or preventing a subject having dilated cardiomyopathy (DCM), comprising (i) genetically modifying a cell from the subject using the method of any one of claims 1 to 27, and (ii) reintroducing the cell into the subject, wherein the reintroducing is effective to treat or prevent the subject having DCM.

In some embodiments, the subject has a point mutation at the RBM20 locus. In some embodiments, the point mutation at the RBM20 locus comprises a substitution at an amino acid position comprising 83, 455, 535, 633, 634, 635, 636, 637, 638, 703, 716, 783, 831, 888, 913, 914, 1031, 1081, 1182, 1206, or a combination thereof; and wherein the substitutions and the positions are determined with reference to SEQ ID NO: 10. In some embodiments, the point mutation at the RBM20 locus comprises a substitution at an amino acid position comprising 633, 634, 635, 636, 637, 638, or a combination thereof. In some embodiments, the point mutation at the RBM20 locus comprises a substitution at amino acid position 633, 634, or a combination thereof. In some embodiments, the substitution comprises P633L, R634Q, or a combination thereof.

In some instances, the cell is reintroduced into the subject by systemic delivery. In other instances, the cell is reintroduced into the subject by local delivery. In some embodiments, the local delivery is intrafemoral or intrahepatic.

In some embodiments, the cell is cultured, expanded, selected, and/or induced to undergo differentiation in vitro prior to being reintroduced into the subject.

In another aspect, the present disclosure provides a sgRNA that specifically targets the RBM20 gene comprising a sequence having about 80% or greater identity to any one of SEQ ID NOs: 1-9.

In another aspect, the present disclosure provides an iPSC comprising such sgRNA and a base editor (BE) comprising an RNA-guided catalytically impaired nuclease fused to a nucleobase deaminase enzyme.

In another aspect, the present disclosure further provides a cardiomyocyte derived from such iPSC.

In another aspect, the present disclosure provides a pharmaceutical composition comprising a plurality of iPSCs disclosed herein, or a plurality of cardiomyocytes disclosed herein.

### BRIEF DESCRIPTION OF THE DRAWINGS

FIG. 1: Molecular and physiological characterization of P635L and R636Q mouse lines. a) Confocal images of isolated adult murine cardiomyocytes. Scale bar: 20 μm. ACTN1 was used as cardiomyocyte marker. b, c) RBM20 granule size (b) and amount (c) in adult mouse cardiomyocytes. N=21 (WT), 16 (P635L HET), 28 (P635L HOM), 16 (R636Q HET) and 39 (R636Q HOM) images with 1-4 cells each obtained from three mice per genotype. Boxplots depict the median with the box including the 25-75^{th} percentile and the whiskers ranging from the smallest to the largest value. d) Number of DEGs (P_{adjust}<0.05) in bulk RNA-seq of Rbm20 mutant mice compared to WT. N=5 mice per genotype. e) GO analysis (biological function) of DEGs overlapping for both P635L and R636Q HOM mice with a stringent cut-off of P_{adjust}<1e⁻¹⁰ to reduce the number of DEGs for display in FIG. 7. f) Number of differentially spliced events compared to WT detected and categorized by rMATS: alternative 5′ or 3′ splice site (A5SS or A3SS), mutually exclusive exons (MXE), retained intron (RI), skipped exon (SE). g) Averaged ΔPSI (percent spliced-in) values relative to WT of significant differentially spliced events (P_{adjust}<0.01, ΔPSI>0.1) overlapping in both HOM Rbm20 mutant mice. Multiple splice events per gene are depicted if they match the selection cut-off. Genes in red were validated by RT-PCR or qPCR. Grey squares indicate that the splice event was not detected by rMATS. h) RT-PCR of RBM20 target genes Tin, Ryr2, Ldb3 and the housekeeping gene Gapdh. i) Kaplan-Meier survival curve of mutant mice monitored for 120 days. P value obtained by Log-rank test between each mutant and WT indicated next to the curves. Percentage of survival indicated for HOM mice. j) Percentage of ejection fraction determined by narcosis echocardiography of mutant mice. N=13 (WT), 5 (P635L HET), 6 (P635L HOM), 11 (R636Q HET) and 11 (R636Q HOM) mice. P values in (b), (c), and (j) obtained from one-way ANOVA with Tukey's multiple comparison test: ****P<0.0001, ***P<0.001, **P<0.01, n.s.=not significant. All data were obtained in 16-week-old mice except in (j) where data of 24-week-old mice is shown. Error bars depict the standard error of the mean (SEM) in all panels.

FIG. 2: Base editing of RBM20 in human iPSC-CMs and in mice. a-c) Transient expression of base editor and gRNA in human iPSCs and iPSC-CMs. Experimental outline in (a), editing efficiency of P633L in (b) and R634Q in (c). “CP” labels the circular permuted base editor CP-1041. Purple line indicates average repair efficiency in iPSC-CMs. d) Generation of stable base editor expression in R634Q iPSCs with a repair efficiency of 34.26±2.36% as determined by amplicon-seq. N=3 independent differentiations. e) Expression of spliced and unspliced isoforms of TTN and IMMT in parental, R634Q and edited R634Q iPSC-CMs differentiated for 15 and 32 days. Significant changes compared to R634Q indicated when present and analyzed by unpaired, two-tailed/tests. *P<0.05, **P<0.01, ****P<0.0001. f) Experimental outline of AAVMYO-mediated base editing in mice. g) Percentage of editing of P635L HOM mice injected with AAVMYO carrying different gRNA-base editor combinations or PBS as empty control. For NRCH-gRNA1, AAV9 was also used as vector. Significance was assessed using unpaired, two-tailed (tests ***P<0.001, **P<0.01, *P<0.05. Sequence shows location of the on-target edit in blue and the two bystander edits in red. Numbers depict the position of the nucleotides within the targeting gRNA (gRNA2 was used as reference) with the PAM sequence in position 21-23. h) Allele frequency of repaired DNA in the muscle tissues heart, diaphragm and quadriceps femoris (quadriceps f.), as well as the liver, plotted for ten mice with the highest editing events in (g). i) Percentage of editing of Rbm20 mRNA in mice treated with AAVMYO-SpRY for 6 or 12 weeks. Editing was assessed by amplicon-seq of cDNA isolated from the whole heart. Most base editors contain the deaminase variant Abemax except when indicated by “8e”, which are base editors with the Abe8e version. Percentage “repaired” in (b, c, g-i) is defined by NGS reads from amplicon-seq with only the wild-type sequence. The number of biological replicates i.e., independent differentiations in (b, c, e) or mice in (g, i) is indicated in brackets above the bars. Error bars depict the SEM in all panels.

FIG. 3: Phenotypic characterization of mice after AAVMYO-ABE treatment. a) Allele frequency of repaired Rbm20 mRNA in mice treated with AAVMYO-ABE determined by RNA-seq. N=3 (R636Q), 4 (P635L) and 8 (WT) mice per condition. b, c) RBM20 staining in whole heart tissue sections in WT and Rbm20 mutant mice treated with PBS or AAVMYO-ABE. Representative images in (b) and quantification of nuclear and cytoplasmic RBM20 localization in (c). Scale bar: 20 μm. Arrows highlight nuclear restored RBM20 (magenta) and in cytoplasmic RBM20 (white) in base edited mice. Manual quantification of >200 nuclei in 2 mice per condition. d) Isoform expression of RBM20 target genes Ttn, Ryr2, Ldb3 and the housekeeping gene Gapdh determined by RT-PCR. N=2-4 mice per condition. e, f) Vertical agarose gel (e) and quantification (f) of titin protein isoforms in WT and Rbm20 mutant mice treated with PBS or AAVMYO-ABE. Based on gel images in FIG. 9d. N=3 mice per condition expect for WT and P635L/SpRY where 4 mice were analyzed. g) RNA-seq data showing changes of the ΔPSI values relative to WT in P635L, or R636Q HOM mice injected with saline or base editor. See method section (bulk RNA sequencing and analysis) for definition of 3 categories. Rescue splice events are labeled in red, all Ttn splice events in blue. N=number of splice events per category. R636Q was sequenced deeper compared to P635L explaining the difference in number of DSGs detected. N=3-5 mice per condition. h-j) Percentage of ejection fraction (h), LVID (i) and cardiac volume (j) determined by narcosis echocardiography of mutant mice treated with PBS or AAVMYO-ABE. N=5 mice per condition. Same WT cohort used as in FIG. 6l-n (16-week time point). P values obtained from one-way ANOVA with Tukey's multiple comparison test: ***P<0.001 **P<0.01, *P<0.05, n.s.=not significant. All data were obtained 12 weeks after AAVMYO-ABB injection. Only homozygous P635L or R636Q mice were treated. Error bars depict the SEM in all panels.

FIG. 4: Cell type-specific profiling of cells after base editing by snRNA-seq. a) UMAP projection integrating all datasets and annotated based on their gene expression profile. b) Expression of known marker genes defining the main cell types. c) UMAP projection of ventricular cardiomyocytes from WT, P635L HOM, and base-edited mice. d) Histograms depicting the distribution of pairwise Euclidean distances of ventricular cardiomyocytes from P635L HOM and base-edited mice relative to WT using the two largest principal components (PC). e) UMAP projection showing the activity score (see ‘Methods’ section snRNA-seq analysis) of cardiomyocytes using a subset of genes that were up- or downregulated in P635L HOM relative to WT. Maximum 15 significantly up- or downregulated were used. f) Threshold of activity score values based on (e) relative to percentage of cells above the threshold for genes upregulated (upper panel) or downregulated (lower panel) in P635L HOM cardiomyocytes relative to WT. g) Percentage of cells above the critical threshold for genes upregulated or downregulated in P635L HOM cells relative to WT. VCMs ventricular cardiomyocytes, ACMs atrial cardiomyocytes, SMCs smooth muscle cells. Data were generated by snRNA-seq of isolated nuclei from two mice per condition.

FIG. 5: WGS of mouse tissue before and after AAVMYO-ABE treatment. a) Mean number of tissue-specific and variants detected in all tissues (common). b) Mean relative distribution of distinct nucleotide conversions for tissue-specific and common SNVs. Tissue overlap represent variants that were common to all three tissues. c) Allele frequency of tissue-specific T>C/A>G variants. N=33 (heart), 32 (liver), 146 (tail). d) Mean number of mismatches to the gRNA and PAM sequence in the area of +30 bases around the variant start site. Error bars depict the SEM in all panels. N=3 mice in (a, b, d).

FIG. 6: Extended characterization of P635L and R636Q mouse lines. a) Sanger sequencing traces of first generation homozygous mutant mice used for subsequent mating and experiments. Red (P635L): C>T mutation; grey (R636Q): GT>AG mutation. Note that for subsequent base editing in R636Q, the CAG codon is converted to CGG which is synonymous to the WT CGT codon. b, c) Expression fold change compared to WT of Rbm20 (b), and Nppa and Nppb (c). mice. d) GO analysis (biological function) of DSGs that overlap for both P635L and R636Q HOM mice with a cut-off of P_{adjust}<0.01 and ΔPSI>0.1. e) Venn diagram of significantly DEGs (up, P_{adjust}<0.05) and DSGs (down, P_{adjust}<0.01 and ΔPSI>0.1). f) P values (cut-off P_{adjust}<0.05) of DEGs unique or common between P635L (red) and R636Q HOM (grey). Significant changes analyzed by unpaired, two-tailed t-tests. ****P<0.0001. Boxplots depict the median with the box including the 25-75^{th} percentile and the whiskers ranging from the smallest to the largest value. The number of genes is shown above the plots. g, h) Expression fold change compared to WT of spliced and unspliced Tin isoforms and Camk2d isoform A (g) or fibrosis marker genes (h) determined by qPCR. Significant changes indicated and analyzed by unpaired, two-tailed t tests. *P<0.05, **P<0.01. i, j) Representative heart tissue sections stained with Sirius Red (i) and quantification of Sirius Red positive area (j). Scale bar: 500 μm. k, 1) Cardiac volume (k) and LVID (1) determined by narcosis echocardiography. Only significant differences are labelled. P values obtained from one-way ANOVA with Tukey's multiple comparison test: **P<0.01. Mice were 24-weeks old. m-o) Percentage of ejection fraction (m), LVID (n) and cardiac volume (o) determined by time-course narcosis echocardiography of Rbm20 mutant mice. Asterisk indicates statistical significance compared to WT obtained by two-way ANOVA with Tukey's multiple comparison test: ****P<0.0001, ***P<0.001, **P<0.01, *P<0.05. Data for the 24 week time point is the same as in FIG. 1j and FIG. 6j, k. Data for R636Q HET and HOM at 12 and 16 weeks has been published by us before⁸². (p) Heart-to-body weight ratio after 24 and 52 weeks of P635L mutant mice. N=5 for HOM and 6 for HET mice unless indicated otherwise in brackets above the bars. No significant changes were found in (j), (1) and (p). All data was obtained in 16-week-old mice if not indicated otherwise. Error bars depict the SEM in all panels. Gene expression analysis was performed using RNA isolated from left ventricles.

FIG. 7: Heatmap of DEGs of P635L and R636Q mice derived from RNAseq. Grey bars indicate that the gene was not detected in RNA-seq data. N=5 mice per genotype. All DEGs that overlap in P635L and R636Q HOM mice are shown with a stringent cut-off of P_{adjust}<1e⁻¹⁰.

FIG. 8: Analysis of base editing in iPSC-CMs and in mice. a-c) Percentage of Indels (a) and bystander edits in P633L (b) and R634Q (c) iPSCs and iPSC-CMs. Indel formation is the summed frequency of insertions or deletions in a window of 10 bp upstream to 10 bp downstream of the gRNA binding site. Indel formation is shown combined for both mutations and gRNAs and separated by different base editors only. Bystander edits are sequences that contain extra A>G conversions within the gRNA window. Observed positions of bystander edits is indicated in red, the on-target site is in blue. d, e) Representative tissue sections (d) and RNA expression data (e) measuring the fluorescent transgene YFP delivered by AAVMYO and injected in different concentrations in WT mice. Scale bar: 100 μm. One mouse per concentration was injected. f) Percentage of repaired reads relative to viral copy number per diploid genome (left) or relative to RNA expression (right) determined by ddPCR in the muscle tissues heart, diaphragm and quadriceps femoris (quadriceps f.), as well as the liver. For the left panel, DNA was used as input with primers for the CMV promoter, for the right panel, RNA reverse transcribed to cDNA was used with primers for the transcribed WPRE element common in all base editor constructs. Only the SpRY-gRNA2 combination was analyzed. Each datapoint represents one mouse. g) Editing efficacy of NRCH/gRNA2 driven by the hTNNT2 or the CAG promoter. Concentration shown is the combined amount of N- and C-terminal base editor-containing AAV. N=2 mice (CAG 1e12 and hTNNT2 2e12, no error bars) or 3 mice (hTNNT2 1e12). h) Editing efficacy of SpRY and 8e-NRCH in heart and liver 6 and 12 weeks after injection in 4-week-old mice. N=3 (8e-NRCH-gRNA2) or 7 (SpRY-gRNA2) mice. i) Normalized RNA expression of RBM20 derived from single-nucleus RNA-seq of the human heart⁸³. SMCs=smooth muscle cells, Vent. CMs=ventricular cardiomyocytes. Error bars depict the SEM in all panels. Only P635L HOM were treated.

FIG. 9: Extended phenotypic characterization of mice after AAVMYOABE treatment. a, b) Allele frequency of repaired DNA (a) and bystander edits (b) in mice treated with AAVMYO-ABE. Sequence shows location of the on-target edit in blue and the bystander edits in red. Numbers depict the position of the nucleotides within the targeting gRNA with the PAM sequence in position 21-23. c) Example allele frequency determined by Crispresso2 for one mouse treated with AAVMYO harboring the base editor 8e-NRCH and gRNA2. On-target location is indicated in blue and location of observed bystander edits in red. d) Expression of spliced and unspliced Tin isoforms and Camk2d isoform A in WT and mutant mice treated with PBS or AAVMYO-ABE. e) Vertical TTN agarose gels showing the G-N2BA, N2BA N2B, and T2 protein isoforms of TTN, and MHC as a loading control. f) Percentage of ejection fraction in WT or mutant mice 8 weeks after injection with PBS or AAVMYO-ABE. P values obtained by one-way ANOVA with Tukey's multiple comparison test shown for AAVMYO versus PBS. N=3-5 mice per condition. g) Expression of heart failure biomarkers Nppa and Nppb in WT and mutant mice treated with PBS or AAVMYO-ABE. h) Ejection fraction before (at week 4) and 8 and 12 weeks after injection of AAVMYO-ABE with hTNNT2-driven NRCH base editor. Twice the amount of AAVMYO-ABE was used compared to CAG-driven ABEs. In contrast to other experiments, we performed echocardiography also before the injection showing that PBS and ABE-treated mice had similar physiological parameters. WT data was obtained separately and is the same as in FIG. 3h. Statistical significance was assessed by unpaired, two-tailed t-tests between PBS and ABE-treated mice. **P<0.01, n.s.=not significant. Only P635L or R636Q HOM mice were treated. Error bars depict the SEM in all panels. The number of mice per condition is indicated in the graphs. All data except in (e) and (g) were obtained 12 weeks after AAVMYO-ABE injection. DNA, RNA and protein isolated from left ventricles.

FIG. 10: Extended snRNA-seq analysis. a-c) Number of active genes (a), total transcript counts (b) and percentage of mitochondrial gene counts per cell (c) for nuclei from each condition. Two independent snRNA-seq experiments were performed for each condition. d) Relative cell type distribution in WT, P635L HOM and base edited mice. e, f) UMAPs (e) and quantification of the fraction of cells expressing the base editor construct (f) delivered by AAVMYO. N- and C-terminal base editor expression values were summed up. Values are either 0 (not expressed, grey) or 1 (expressed, red). g) Histograms depicting the distribution of pairwise Euclidean distances of depicted cell types from P635L HOM and base edited mice relative to WT upon mapping using two principal components (PC). h) Threshold of activity score of depicted cell types (see methods for calculation) relative to percentage of cells above the threshold for genes upregulated (upper panel) or downregulated (lower panel) in P635L HOM relative to WT.

FIG. 11: AAV coverage and editing events detected by WGS. a) Normalized read coverage across autosomes of C- and N-terminal base editor sequence delivered by AAVMYO. b) Allele frequency of the P635L A>G nucleotide conversion. c, d) All (left) and novel (right) SNVs (c) or Indels (d) called by four variant callers (HC: HaplotypeCaller, MT: Mutect2, LF: Lofreq, SC: Scalpel). e) Mean relative distribution of tissue-specific and common variants within coding or non-coding regions of the genome. Tissue overlap represent variants that were common to all three tissues. f) Allele frequency of 16 candidate loci analyzed by amplicon-seq. Seven loci were determined by in silico predictions of off-target editing based on gRNA sequence similarity. Nine loci were obtained from WGS by taking A>G/T>C SNVs with the coverage. N=5 mice treated with PBS and 5 mice treated with AAVMYO-SpRY. Error bars depict the SEM in all panels.

FIG. 12: RNA on- and off-target editing. a) Base editor expression in heart and liver tissue 12 weeks after AAVMYO-ABE or PBS treatment in P635L or P636Q HOM mice. RPKM=reads per kilobase million. b) On-target edit (blue arrow) and bystander edit (red arrow) within the gRNA region targeting Rbm20 on chromosome 19. Shown is the percentage of each base aligned to the positions plotted on the x-axis for R636Q and P635L HOM mice. In each row, three replicated were summed before calculating the fractions. c) Number of heart (H), liver (L) and common variants after filtering as described in the method section. d) Averaged relative amount of distinct types of SNVs identified as heart-specific. Significance tests were performed with a logistic regression model using the python package statsmodels and testing the difference in A<G vs. non-A<G ratios between pairs of ABE and PBS-treated samples. Error bars depict standard deviation. e) Number of mismatches to the gRNA and PAM sequence in the area of +30 bases around the variant start site. Variants from three replicates were summed up. Variants on the X or Y chromosome were excluded. Error bars depict the SEM in all panels. Box plots in (a) and (c) depict the median including the 25th-75th percentile with whiskers extending to the rest of the distribution.

### DETAILED DESCRIPTION

### Introduction

Provided herein are compositions and methods for treating or preventing dilated cardiomyopathy (DCM) in a subject in need thereof, e.g., through correcting one or more point mutations in the genomic DNA of cells taken from a subject, and subsequently reintroducing the genetically modified cells back into the subject. In one particular aspect, the disclosure describes the use of a CRISPR-associated base editing to correct point mutations at the RBM20 locus in a cell. For the first time, the inventors employ a combination of the viral vector AAVMYO with targeting specificity of heart muscle tissue and CRISPR base editors (BEs) to repair DCM patient mutations in the cardiac splice factor RBM20, demonstrating the potential of base editors combined with AAVMYO to achieve gene repair for treatment of DCM and other hereditary cardiac diseases. Furthermore, the inventors use the intein-mediated trans-splicing strategy to package the gRNA/BE complex in dual recombinant adeno-associated viruses (rAAVs), to optimize viral dosages inside of the cells thus enhancing base editing efficiency.

### Definitions

Before the present invention is further described, it is to be understood that this invention is not strictly limited to particular embodiments described, as such may of course vary. It is also to be understood that the terminology used herein is for the purpose of describing particular embodiments only, and is not intended to be limiting, since the scope of the present invention will be limited only by the claims.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells, and so forth.

The terms “about” and “approximately” as used herein shall generally mean an acceptable degree of error for the quantity measured given the nature or precision of the measurements. Typically, exemplary degrees of error are within 20 percent (%), preferably within 10%, and more preferably within 5% of a given value or range of values. Any reference to “about X” specifically indicates at least the values X, 0.8X, 0.81X, 0.82X, 0.83X, 0.84X, 0.85X, 0.86X, 0.87X, 0.88X, 0.89X, 0.9X, 0.91X, 0.92X, 0.93X, 0.94X, 0.95X, 0.96X, 0.97X, 0.98X, 0.99X, 1.01X, 1.02X, 1.03X, 1.04X, 1.05X, 1.06X, 1.07X, 1.08X, 1.09X, 1.1X, 1.11X, 1.12X, 1.13X, 1.14X, 1.15X, 1.16X, 1.17X, 1.18X, 1.19X, and 1.2X. Thus, “about X” is intended to teach and provide written description support for a claim limitation of, e.g., “0.98X.”

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “gene” means the segment of DNA involved in producing a polypeptide chain. It may include regions preceding and following the coding region (leader and trailer) as well as intervening sequences (introns) between individual coding segments (exons).

A “promoter” is defined as an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. The promoter can be a constitutive promoter, such as a CAG promoter, which is active in a cell under all circumstances. The promoter can also be a regulatory promoter or inducible promoter, which becomes activated under specific circumstances, such as a chemically inducible promoter, a temperature inducible promoter, a light inducible promoter, etc. In some embodiments, the promoter can be only activated in particular organs/tissues, such as a human cardiac troponin T (hTNNT2) promoter, which is specifically activated in heart but not in other tissues/organs.

“Polypeptide,” “peptide,” and “protein” are used interchangeably herein to refer to a polymer of amino acid residues. All three terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins, wherein the amino acid residues are linked by covalent peptide bonds.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used in herein, the terms “identical” or percent “identity,” in the context of describing two or more polynucleotide or amino acid sequences, refer to two or more sequences or specified subsequences that are the same. Two sequences that are “substantially identical” have at least 60% identity, preferably 65%, 70%, 75%, 80%, 85%, 90%, 91%, 92%, 93%, 94%, 95%, 96%, 97%, 98%, 99%, or 100% identity, when compared and aligned for maximum correspondence over a comparison window, or designated region as measured using a sequence comparison algorithm or by manual alignment and visual inspection where a specific region is not designated. With regard to polynucleotide sequences, this definition also refers to the complement of a test sequence. With regard to amino acid sequences, in some cases, the identity exists over a region that is at least about 50 amino acids or nucleotides in length, or more preferably over a region that is 75-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST 2.0 algorithm and the default parameters discussed below are used.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned.

An algorithm for determining percent sequence identity and sequence similarity is the BLAST 2.0 algorithm, which is described in Altschul et al., (1990) J. Mol. Biol. 215:403-410. Software for performing BLAST analyses is publicly available at the National Center for Biotechnology Information website, ncbi.nlm.nih.gov. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=˜2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The “CRISPR-Cas” system refers to a class of bacterial systems for defense against foreign nucleic acids. CRISPR-Cas systems are found in a wide range of bacterial and archaeal organisms. CRISPR-Cas systems fall into two classes with six types, I, II, III, IV, V, and VI as well as many sub-types, with Class 1 including types I and III CRISPR systems, and Class 2 including types II, IV, V and VI; Class 1 subtypes include subtypes I-A to I-F, for example. See, e.g., Fonfara et al., Nature 532, 7600 (2016); Zetsche et al., Cell 163, 759-771 (2015); Adli et al. (2018). Endogenous CRISPR-Cas systems include a CRISPR locus containing repeat clusters separated by non-repeating spacer sequences that correspond to sequences from viruses and other mobile genetic elements, and Cas proteins that carry out multiple functions including spacer acquisition, RNA processing from the CRISPR locus, target identification, and cleavage. In class 1 systems these activities are effected by multiple Cas proteins, with Cas3 providing the endonuclease activity, whereas in class 2 systems they are all carried out by a single Cas, Cas9.

The term “treating” or “treatment” refers to any one of the following: ameliorating one or more symptoms of a disease or condition (e.g., a dilated cardiomyopathy); slowing down or completely terminating the progression of the disease or condition (as may be evident by longer periods between reoccurrence episodes, slowing down or prevention of the deterioration of symptoms, etc.); enhancing the onset of a remission period; slowing down the irreversible damage caused in the progressive-chronic stage of the disease or condition (both in the primary and secondary stages); delaying the onset of said progressive stage; or any combination thereof.

The term “prevent” or “preventing” refers protecting a subject that is at risk for a disease or condition (e.g., a dilated cardiomyopathy) from developing the disease or condition, or decreasing the risk that a subject can develop the disease or condition.

As used herein, the terms “subject”, “individual” or “patient” refer, interchangeably, to a warm-blooded animal such as a mammal. In particular embodiments, the term refers to a human. A subject may have, be suspected of having, or be predisposed to a lysosomal storage disorder as described herein. The term also includes livestock, pet animals, or animals kept for study, including horses, cows, sheep, poultry, pigs, cats, dogs, zoo animals, goats, primates (e.g. chimpanzee), and rodents. A “subject in need thereof” refers to a subject that has one or more symptoms of dilated cardiomyopathy (DCM), that has received a diagnosis of a DCM, that is suspected of having or being predisposed to a DCM, and/or that has identified one or more point mutations of DCM-associated genes.

As used herein, the term “administering” includes oral administration, topical contact, administration as a suppository, intravenous, intraperitoneal, intramuscular, intralesional, intratumoral, intradermal, intralymphatic, intrathecal, intranasal, or subcutaneous administration to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc.

The term “pharmaceutically acceptable carrier” refers to a substance that aids the administration of an active agent to a cell, an organism, or a subject. “Pharmaceutically acceptable carrier” refers to a carrier or excipient that can be included in the compositions of the disclosure and that causes no significant adverse toxicological effect on the subject. Non-limiting examples of pharmaceutically acceptable carriers include water, sodium chloride, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g. antimicrobial preservatives, antioxidants, chelating agents and buffers), for preventing the action of microorganisms (e.g. antimicrobial and antifungal agents, such as parabens, chlorobutanol, sorbic acid and the like) or for providing the formulation with an edible flavor etc. In some instances, the carrier is an agent that facilitates the delivery of a modified cell to a target cell or tissue. One of skill in the art will recognize that other pharmaceutical carriers are useful in the present disclosure.

### Dilated Cardiomyopathy

The present disclosure provides methods and compositions for treating or preventing dilated cardiomyopathy (DCM). DCM is a heterogeneous disease with multiple causes and a non-specific phenotype that ultimately leads to the dilation of the left ventricle and systolic dysfunction. Approximately 30% of DCM is due to inherited mutations of several structural components of the heart, and 19 genes are classified for a strong connection to DCM: RBM20 (encoding RNA binding motif protein 20), BAG3 (BLC2-associated athanogene 3), DES (desmin), FLNC (filamin-C), LMNA (lamin A/C), MYH7 (myosin heavy chain 7), PLN (phospholamban), SCN5A (sodium channel α-subunit), TNNC1 (troponin C), TNNT2 (troponin T), TIN (titin), DSP (desmoplakin), ACTC1 (cardiac α-actin), ACTN2 (α-actinin-2), JPH2 (Junctophilin 2), NEXN (nexilin), TNNI3 (troponin I), TPM1 (α-tropomyosin), and VCL (vinculin). As disclosed herein, the methods and compositions can be used to correct point mutations in any of these genes. In some embodiments, the method disclosed herein is used to correct one or more point mutations in a gene selected from the group consisting of RBM20, BAG3, DES, FLNC, LMNA, MYH7, PLN, SCN5A, TNNC1, TNNT2, TTN, DSP, ACTC1, ACTN2, JPH2, NEXN, TNNI3, TPM1, and VCL.

### RBM20

In particular embodiments, the methods and compositions disclosed herein can be used to correct point mutations at the RBM20 locus. The RBM20 gene encodes RNA-binding motif protein 20 that regulates RNA splicing of genes critical for the function of cardiomyocytes. The human RBM20 gene is located on the long arm of chromosome 10 and carries 14 exons. It encodes a 1227 amino acid protein containing two zinc finger domains, a glutamate-rich region, a leucine-rich region, an RNA-Recognition Motif (RRM)-type RNA binding domain and an arginine-/serine-rich region (RS-domain). Human RBM20 protein comprises a sequence of SEQ ID NO: 10.

Pathogenic variants in RBM20 account for approximately 2-6% of the cases of familial DCM with noticeably early disease onset and clinically severe expression. Three protein regions were identified with high confidence for carrying pathogenic variants. These are located at positions c.1601-1640 (exon 7, encoding the RRM-domain), c.1881-1920 (exon 9, encoding the highly conserved RS-domain) and c.2721-2760 (exon 11). Table 1 presents reported variants with corresponding domains. In particular, the BM20 mutations enriched in a small stretch of six amino acids: Proline-Arginine-Serine-Arginine-Serine-Proline (PRSRSP) within the RS-domain result in aberrant formation of cytoplasmic granules and amplify DCM-specific disease phenotype.

As disclosed herein, the methods and compositions for treating or preventing DCM can be used to correct any point mutation on the RBM20 gene. In some embodiments, the RBM20 point mutation comprises a substitution at an amino acid position comprising 83, 455, 535, 633, 634, 635, 636, 637, 638, 703, 716, 783, 831, 888, 913, 914, 1031, 1081, 1182, 1206, or a combination thereof; and wherein the substitutions and the positions are determined with reference to SEQ ID NO: 10. In some embodiments, the point mutation at the RBM20 locus comprises a substitution at an amino acid position within the six amino acid stretch (PRSRSP) comprising 633, 634, 635, 636, 637, 638, or a combination thereof. In particular embodiments, the point mutation at the RBM20 locus comprises a substitution at amino acid position 633, 634, or a combination thereof.

In some embodiments, the RBM20 point mutation to be corrected is selected from the group consisting of L831, S455L, V535I, P633L, R634Q, R634W, S635A, R636C, R636H, R636S, S637G, P638L, R703S, R716Q, R783G, L831I, D888N, E913K, V914A, G1031X, P1081R, R1182H, E1206K, or a combination thereof. In some embodiments, the RBM20 point mutations of interest are enriched in the six amino acid stretch (PRSRSP) within the RS-domain, including P633L, R634Q, R634W, S635A, R636C, R636H, R636S, S637G and P638L. In particular embodiments, the RBM20 point mutation comprises P633L, R634Q, or a combination thereof.

TABLE 1
RBM20 variants with corresponding exons and protein domains.

| Domain | Mutation | Exon |
|---|---|---|
| Leu-rich-region | L83I | 2 |
| Other | S455L | 4 |
| RRM-domain | V535I | 6 |
| RS-domain | P633L | 9 |
| RS-domain | R634Q | 9 |
| RS-domain | R634W | 9 |
| RS-domain | S635A | 9 |
| RS-domain | R636C | 9 |
| RS-domain | R636H | 9 |
| RS-domain | R636S | 9 |
| RS-domain | S637G | 9 |
| RS-domain | P638L | 9 |
| Other | R703S | 9 |
| Other | R716Q | 9 |
| Other | R783G | 9 |
| Other | L831I | 11 |
| Glu-rich-region | D888N | 11 |
| Glu-rich-region | E913K | 11 |
| Glu-rich-region | V914A | 11 |
| Other | G1031X * | 11 |
| Other | P1081R | 11 |
| ZnF-2 | R1182H | 13 |
| ZnF-2 | E1206K | 13 |

* non-sense mutation; all others are missense mutations.

### Base Editor (BE)

As disclosed herein, the present methods and compositions for correcting point mutations involve CRISPR-associated base editing. Base editing is a CRISPR-based genome editing technology that allows the introduction or correction of point mutations in the DNA without generating double-strand breaks (DSBs). Base editors (BEs) can be cytidine base editors (CBEs) allowing C>T conversions or adenine base editors (ABEs) allowing A>G conversions.

BEs used for correcting point mutations can be any kinds of BEs known in the art. In some instances, the BE is an adenine base editor (ABE). In other instances, the BE is a cytidine base editor (CBE). In some embodiments, the BE is selected from the group consisting of BE1, BE2, BE3, BE4, BE4max, AncBE4max, ABE6.3, ABE7.8, ABE7.9, ABE7.10, ABEmax, ABE8e, ABE-SpRY, CBE-SpRY, ABE-CP-1041, and CBE-CP-1041.

### Cytosine Base Editor (CBE)

The early generations of BEs, including BE1, BE2, BE3, and BE4, are all CBEs converting a G:C bp to T: A bp. BE1 is the first-generation BE comprising a catalytically dCas9 from Streptococcus pyogenes (Sp) fused with a rat deaminase (rAPOBEC1). The dCas9 contains the D10A and H840A amino acid substitutions of Cas9 that abolish the nuclease activity avoiding DSB generation without interfering with its DNA binding capacity. BE2 is based on BE1 further fused with an uracil glycosylase inhibitor (UGI) to the dCas9 to protect newly formed U from excision. BE3 is the third generation of BE replacing the dCas9 of BE2 with a Cas9 nickase (Cas9n containing the D10A amino acid substitution) that nicks the non-edited G-containing DNA strand without generating DSBs. BE4 differs from BE3 as it carries a second UGI conferring a higher editing efficiency and improved product purity.

CBEs can be further optimized by modifying the codon usage and the nuclear localization sequences to enhance base editing in mammalian cells (e.g., BE4max, and AncBE4max). For instance, BE4 can be improved by the addition of a bipartite NLS at both N- and C-termini and by codon optimization to generate BE4max. Replacement of rAPOBEC1 with an optimized ancestor rAPOBEC1 homolog—Anc689 that contains 36 amino acid substitutions compared to rAPOBEC1—resulted in the generation of AncBE4max. Both BE4max and AncBE4max exhibit a higher editing efficiency compared to BE4.

### Adenine Base Editor (ABE)

As disclosed herein, an ABE allows an A:T bp to a G:C bp conversion at a target locus (e.g., RBM20). In some embodiments, the ABE comprises a catalytically impaired nuclease and an adenine deaminase. In some instances, the adenosine deaminase is a dimeric adenine deaminase. In some embodiments, the dimeric adenine deaminase is a heterodimer comprising a wild-type tRNA adenosine deaminase (TadA) and a genetically modified TadA*. As disclosed herein, the genetically modified TadA* comprises one or more (e.g., 1, 2, 3, 4, 5, 6, 7, 8, 9, 10, or more) substitutions of the wild-type TadA amino acid sequence. As disclosed herein, the genetically modified TadA* can deaminates an adenine in a DNA sequence. In some embodiments, the dimeric adenine deaminase is a homodimer comprising two genetically modified TadAs*. In some embodiments, the adenosine deaminase is a monomer adenine deaminase comprising one genetically modified TadA*. ABE6.3, ABE7.8, ABE7.9, and ABE7.10, and ABE8e are examples of ABEs with different mutations in the TadA* domain. In some embodiments, the ABE is selected from the group consisting of ABE6.3, ABE7.8, ABE7.9, ABE7.10, ABEmax, ABE8e, ABE-SpRY, and ABE-CP-1041.

ABEs can be optimized by using orthologous or engineered Cas9n to broaden the range of adenine base editing targets. For instances, Cas9n variants can be introduced in ABEs to generate A>G conversions at genomic sites containing non-NGG PAMs. In some instances, SpCas9n is replaced by SaKKHn or SpCas9n-VQR in ABE7.10 and generates SaKKH-ABE and VQR-ABE, respectively. In some instances, xCas9 is introduced in ABE7.10 to generate xCas9-ABE. ABEmax versions contain Cas9 variants recognizing NG (xCas9 in xABEmax or SpCas9n-NG in NG-ABE max) or NR PAMs (SpCas9n-NRCH, SpCas9n-NRTH, and SpCas9n-NRRH). ABEmax can be further improved by replacing SpCas9n with SaCas9n or with the engineered SaKKHn, SpCas9n-VRER and SpCas9n-VRQR allowing the targeting of loci containing non-NGG PAMs. SpCas9n-VRER and SpCas9n-VRQR induce A-to-G conversions in many target sites containing PAMs other than NGG. Sa-ABEmax and SaKKH-ABEmax present a large editing window (position 4-14 of the protospacer). CP-ABEmax can target bases located outside the canonical editing window.

In addition, ABEs can be optimized by modifying TadA* to enhance adenine base editing in cells. ABE8e contains eight additional mutations in the TadA* deaminase domain that confer a higher processing activity. ABE8e further increases editing efficiency when combined with SpCas9n or different Cas9 variants (e.g., SaCas9n, SaKKHn, SpCas9n-NG, and LbCas12a) compared to the corresponding ABEmax-based enzymes. Furthermore, removal of the wild type TadA did not affect ABE8e editing activity, indicating that the optimized TadA* can efficiently work as a monomer.

As disclosed herein, a base editor (BE) typically comprises two components: a nucleobase deaminase enzyme and an RNA-guided catalytically impaired nuclease. The two components can be linked covalently (e.g., as a fusion protein) or non-covalently (e.g., through an RNA aptamer). The catalytically impaired nuclease, guided by a single guide RNA (sgRNA), recognizes a specific sequence named protospacer adjacent motif (PAM) and unwinds the DNA sequence upstream of the PAM (“protospacer”). Thus, the deaminase enzyme converts the bases located in a specific DNA stretch of the protospacer “editing window”.

### Nucleobase Deaminase Enzyme

The nucleobase deaminase enzyme can chemically modify a specific DNA base. It can convert one nucleotide into another by catalyzing the removal of an amino group from the base. For example, cytosine can be converted into uracil, which can ultimately lead to a base pair change from C-G to T-A. In some embodiments, the nucleobase deaminase enzyme is a single-stranded DNA (ssDNA)-specific nucleobase deaminase enzyme. In some instances, the deaminase enzyme is an adenine deaminase. In other instances, the deaminase enzyme is a cytidine deaminase.

The cytosine deaminase used in the CBEs can be a rat deaminase (rAPOBEC1), a rAPOBEC1 variant (evoAPOBEC1), an ancestor of rAPOBEC1 (EvoFERNY), an optimized ancestor rAPOBEC1 homolog (Anc689), a P. marinus activation-induced cytidine deaminase (AID or PmCDA1), a PmCDA1 variant (evoCDA1), a human APOBEC3A (hA3A), or any variant thereof.

The adenine deaminase used in the ABEs comprises a genetically modified TadA*. As disclosed herein, the genetically modified TadA* can deaminates an adenine in a DNA sequence. As disclosed herein, the genetically modified TadA* comprises one or more (e.g., 1, 2, 3, 4, 5, 6, 7, 8, 9, 10, or more) substitutions of the wild-type TadA amino acid sequence. In some instances, the adenosine deaminase is a dimeric adenine deaminase. In some embodiments, the dimeric adenine deaminase is a heterodimer comprising a wild-type tRNA adenosine deaminase (TadA) and a genetically modified TadA*. In some embodiments, the dimeric adenine deaminase is a homodimer comprising two genetically modified TadAs*. In some embodiments, the adenosine deaminase is a monomer adenine deaminase comprising one genetically modified TadA*.

### RNA-Guided Catalytically Impaired Nuclease

The RNA-guided catalytically impaired nuclease guides the base editor to the specific location in the DNA where the desired base change should occur. In some embodiments, the RNA-guided catalytically impaired nuclease is a catalytically impaired CRISPR associated (Cas) nuclease, such as a dead Cas9 (dCas9), a dead Cas12 (dCas12), a Cas9 nickase (Cas9n), or a derivative thereof.

The Cas9n or other catalytically impaired nuclease used in the present methods can be from any source, so long that it is capable of binding to an sgRNA of the invention and being guided to the specific sequence (e.g., RBM20 locus) targeted by the targeting sequence of the sgRNA. In some embodiments, the catalytically impaired Cas nuclease is derived from Streptococcus pyogenes (Sp), Staphylococcus aureus (Sa), Staphylococcus auricularis (Sauri), Acidaminococcus sp. (As), Streptococcus macacae (Spy mac), or other bacteria. In particular embodiments, the Cas9n or other catalytically impaired nuclease is from Streptococcus pyogenes.

In some embodiments, the catalytically impaired Cas nuclease recognizes non-NGG PAMs. In some embodiments, the catalytically impaired Cas nuclease is selected from the group consisting of SpCas9n-NRNH (NRNH PAM), SpCas9n-NRTH (NRTH PAM), SpCas9n-NRCH (NRCH PAM), SpRY (NRN and NYN PAM), CP-1041, SpCas9n-VQR (NGA PAM), SpCas9n-VRQR (NGA PAM), SpCas9n-EQR (NGAG PAM), SpCas9n-VRER (NGCG PAM), SaCas9n (NNGRRT PAM), SaCas9n-KKH (SaKKHn) (NNNRRT PAM), SauriCas9n (NNGG PAM), Spy-macCas9n (TAAA PAM), xCas9 (NG, GAA, and GAT PAM), SpCas9n-NG (NG PAM), dLbCas12a (dLbCpf1) (TTTV PAM), and enAsCas12a (TTYN, VTTV, TRTV, or TTTV PAM).

In particular embodiments, the catalytically impaired Cas nuclease is a Cas9n or a derivative thereof. In some embodiments, the Cas9n is an engineered Cas9n. In some embodiments, the engineered Cason is selected from the group consisting of Cas9n-NRNH, Cas9n-NRTH, Cas9n-NRCH, SpRY, CP-1041, Cas9n-VQR, Cas9n-VRQR, Cas9n-EQR, Cas9n-VRER, SaCas9n, SaCas9n-KKH (SaKKHn), SauriCas9n, Spy-macCas9n, xCas9, and Cas9n-NG. In particular embodiments, the engineered Cas9n is Cas9n-NRNH, Cas9n-NRTH, Cas9n-NRCH, CP-1041, or SpRY.

sgRNA

The single guide RNAs (sgRNAs) can target the RBM20 gene or any other genes associated to DCM. sgRNAs interact with a catalytically impaired nuclease such as Cas9n, and specifically bind to or hybridize to a target nucleic acid within the genome of a cell, such that the sgRNA and the site-directed catalytically impaired nuclease co-localize to the target nucleic acid in the genome of the cell. The sgRNAs as used herein comprise a targeting sequence comprising homology (or complementarity) to a target DNA sequence, and a constant region that mediates binding to the RNA-guided catalytically impaired nuclease.

In one aspect, the sgRNA targets at the RBM20 locus. In some embodiments, the sgRNA targets within exon 7, exon 9, or exon 11 of RBM20. In some embodiments, the sgRNA targets within the RS domain at exon 9 of RBM20. In some embodiments, the sgRNA targets the six amino acid stretch (PRSRSP) within the RS domain. In some embodiments, the sgRNA specifically targets the RBM20 gene comprising a sequence having about 80% or greater identity to any one of SEQ ID NOs: 1-9. In some embodiments, the sgRNA comprises a sequence having, e.g., at least 80%, 81%, 82%, 83%, 84%, 85%, 86%, 87%, 88%, 89%, 90%, 91%, 92%, 93%, 94%, 95%, 96%, 97%, 98%, 99%, or more identity to any one of SEQ ID NOs: 1-9, or comprising, e.g., 1, 2, 3 or more nucleotide substitutions in any one of SEQ ID NOs: 1-9. In particular embodiments, the sgRNA comprises a sequence of any one of SEQ ID NOs: 1-9.

The targeting sequence of the sgRNAs may be, e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length, or 15-25, 18-22, or 19-21 nucleotides in length, and shares homology with a targeted genomic sequence, in particular at a position adjacent to a CRISPR PAM sequence. The sgDNA targeting sequence is designed to be homologous to the target DNA, i.e., to share the same sequence with the non-bound strand of the DNA template or to be complementary to the strand of the template DNA that is bound by the sgRNA. The homology or complementarity of the targeting sequence can be perfect (i.e., sharing 100% homology or 100% complementarity to the target DNA sequence) or the targeting sequence can be substantially homologous (i.e., having less than 100% homology or complementarity, e.g., with 1-4 mismatches with the target DNA sequence).

Each sgRNA also includes a constant region that interacts with or binds to the site-directed nuclease, e.g., Cas9n. In the nucleic acid constructs provided herein, the constant region of an sgRNA can be from about 70 to 250 nucleotides in length, or about 75-100 nucleotides in length, 75-85 nucleotides in length, or about 80-90 nucleotides in length, or 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or more nucleotides in length. The overall length of the sgRNA can be, e.g., from about 80-300 nucleotides in length, or about 80-150 nucleotides in length, or about 80-120 nucleotides in length, or about 90-110 nucleotides in length, or, e.g. 90, 91, 92, 93, 94, 95, 96, 97, 98, 99, 100, 101, 102, 103, 104, 105, 106, 107, 108, 109, or 110 nucleotides in length.

It will be appreciated that it is also possible to use two-piece gRNAs (cr:tracrRNAs) in the present methods, i.e., with separate crRNA and tracrRNA molecules in which the target sequence is defined by the crispr RNA (crRNA), and the tracrRNA provides a binding scaffold for the Cas nuclease.

The sgRNAs can be obtained in any of a number of ways. For sgRNAs, primers can be synthesized in the laboratory using an oligo synthesizer, e.g., as sold by Applied Biosystems, Biolytic Lab Performance, Sierra Biosystems, or others. Alternatively, primers and probes with any desired sequence and/or modification can be readily ordered from any of a large number of suppliers, e.g., ThermoFisher, Biolytic, IDT, Sigma-Aldritch, GeneScript, etc.

### Intein-Mediated Trans-Splicing

As disclosed herein, the sgRNA and the BE can be introduced into a cell in one or more expression cassettes. In some embodiments, the sgRNA and the BE are present together in one expression cassette. In some embodiments, the sgRNA and the BE are present separately, in two expression cassettes. In some embodiments, the BE is present in one expression cassette. In some embodiments, the BE is present in two or more expression cassettes, wherein an active BE is packaged in the cell through intein-mediated trans-splicing.

The term “intein-mediated trans-splicing” refers to an autocatalytic process where two protein fragments are joined together to create a functional protein using a naturally occurring or engineered intein. The term “intern” or “protein intron” refers to a segment of a protein that is able to excise itself and join the remaining portions (the exteins) with a peptide bond during protein splicing. In some embodiments, the BE is present in two or more expression cassettes, and packaged through intein-mediated trans-splicing. For example, the N-terminal half of the BE protein is fused to the N-terminal halves of an intein in one expression cassette, and C-terminal half of the BE protein is fused to the C-terminal halves of the intein in the other expression cassettes. After intein-mediated trans-splicing, the N-terminal half of the BE is linked with the C-terminal half of the BE resulting a functional BE useful for the present invention. The expression cassettes disclosed herein are typically driven by a promoter. In some instances, the promoter is a constitutive promoter, such as a CAG promoter. In other instances, the promoter is a muscle-specific promoter, such as a human cardiac troponin T (hTNNT2) promoter or a SPc5-12 promoter.

### Delivery Methods

The sgRNA and BE can be introduced into a cell using any suitable method, e.g., by introducing one or more polynucleotides encoding the sgRNA and the BE into the cell, e.g., using a vector such as a viral vector or delivered as naked DNA or RNA, such that the sgRNA and BE are expressed in the cell. In some instances, the sgRNA and/or the BE are introduced into the cell using a recombinant adeno-associated virus (rAAV) vector. In other instances, the sgRNA and/or the BE are introduced into the cell as a ribonucleoprotein (RNP).

rAAV

The rAAV can be from serotype 1 (e.g., an rAAV1 vector), 2 (e.g., an rAAV2 vector), 3 (e.g., an rAAV3 vector), 4 (e.g., an rAAV4 vector), 5 (e.g., an rAAV5 vector), 6 (e.g., an rAAV6 vector), 7 (e.g., an rAAV7 vector), 8 (e.g., an rAAV8 vector), 9 (e.g., an rAAV9 vector), 10 (e.g., an rAAV10 vector), or 11 (e.g., an rAAV11 vector). In some embodiments, the vector is an rAAV9 vector or a derivative thereof. In particular embodiments, the vector is an AAVMYO, an rAAV9 mutant specifically targeting muscle cells such as cardiomyocytes.

### Ribonucleoprotein (RNP)

In some embodiments, the sgRNA and BE are assembled into ribonucleoproteins (RNPs) prior to delivery to the cells, and the RNPs are introduced into the cell by, e.g., electroporation. RNPs are complexes of RNA and RNA-binding proteins. In the context of the present methods, the RNPs comprise the BE (e.g., ABE) assembled with the guide RNA (e.g., sgRNA), such that the RNPs are capable of binding to the target DNA (through the sgRNA component of the RNP) and modifying it (via the BE component of the RNP). As used herein, an RNP for use in the present methods can comprise any of the herein-described guide RNAs and any of the herein-described base editors.

### Cells

Animal cells, mammalian cells, preferably human cells, modified ex vivo, in vitro, or in vivo are contemplated in the present disclosure. Also included are cells of other primates; mammals, including commercially relevant mammals, such as cattle, pigs, horses, sheep, cats, dogs, mice, rats; birds, including commercially relevant birds such as poultry, chickens, ducks, geese, and/or turkeys.

In some embodiments, the cell is an embryonic stem cell, a stem cell, a progenitor cell, a pluripotent stem cell, an induced pluripotent stem (iPS) cell, a somatic stem cell, a differentiated cell, a mesenchymal stem cell or a mesenchymal stromal cell, a neural stem cell, a hematopoietic stem cell or a hematopoietic progenitor cell, an adipose stem cell, a keratinocyte, a skeletal stem cell, a muscle stem cell, a fibroblast, an NK cell, a B-cell, a T cell, a peripheral blood mononuclear cell (PBMC), or any derivative thereof. In some embodiments, the cell is an iPSC. In some embodiments, the cell is a cardiomyocyte derived from the iPSC (CM-iPSC).

Disclosed herein, in some embodiments, further includes a modified cell comprising an sgRNA and a base editor (BE) comprising an RNA-guided catalytically impaired nuclease fused to a nucleobase deaminase enzyme. In some embodiments, the modified cell is an iPSC. In some embodiments, the modified cell is a cardiomyocyte derived from an iPSC.

### Method of Treating or Preventing DCM

Following the delivering the sgRNA and BE into the cell, e.g., iPSC, and confirming correct modification of the target gene in the cell, a plurality of modified cells can be reintroduced into the subject, such that they can repopulate and differentiate into, e.g., cardiomyocytes, and due to the correction of the target point mutation(s), can treat or prevent one or more abnormalities or symptoms in the subject having dilated cardiomyopathy (DCM). In some embodiments, the cells are cultured, expanded, selected, or induced to undergo differentiation in vitro prior to reintroduction into the subject.

Disclosed herein, in some embodiments, are methods of treating or preventing an DCM in an individual in need thereof, the method comprising correcting one or more point mutations (PMs) causing the DCM in the individual using the genome modification methods disclosed herein. In some instances, the method comprises reintroducing the modified cell, comprising an BE and a sgRNA specifically targeting a sequence comprising the point mutation(s), i.e., at the RBM20 locus, wherein said modified cell comprises correct nucleotide and amino acid sequence, thereby treating or presenting the DCM in the individual.

In some embodiments, the subject has a point mutation at the RBM20 locus. In some instances, the point mutation at the RBM20 locus comprises a substitution at an amino acid position comprising 83, 455, 535, 633, 634, 635, 636, 637, 638, 703, 716, 783, 831, 888, 913, 914, 1031, 1081, 1182, 1206, or a combination thereof; and wherein the substitutions and the positions are determined with reference to SEQ ID NO: 10. In some instances, the point mutation at the RBM20 locus comprises a substitution at an amino acid position comprising 633, 634, 635, 636, 637, 638, or a combination thereof. In some instances, the point mutation at the RBM20 locus comprises a substitution at amino acid position 633, 634, or a combination thereof. In particular instances, the substitution comprises P633L, R634Q, or a combination thereof.

The modified cells of the present disclosure may be administered by any delivery route, systemic delivery or local delivery. These include, but are not limited to, enteral, gastroenteral, epidural, oral, transdermal, intracerebral, intracerebroventricular, epicutaneous, intradermal, subcutaneous, nasal, intravenous, intra-arterial, intramuscular, intracardiac, intraosseous, intrathecal, intraparenchymal, intraperitoneal, intravesical, intravitreal, intracavernous), interstitial, intra-abdominal, intralymphatic, intramedullary, intrapulmonary, intraspinal, intrasynovial, intrathecal, intratubular, parenteral, percutaneous, periarticular, peridural, perineural, periodontal, rectal, soft tissue, and topical. In some instances, the modified cells are reintroduced into the subject by systemic delivery. In other instances, the modified cells are reintroduced into the subject by local delivery. In some embodiments, the local delivery is intrafemoral or intrahepatic.

### Pharmaceutical Compositions

Disclosed herein, in some embodiments, are pharmaceutical compositions comprising a plurality of genetically modified cells through base editing.

In some embodiments, a pharmaceutical composition comprises a plurality of genetically modified iPSCs or CM-iPSCs disclosed herein. The pharmaceutical composition can further comprise a pharmaceutically acceptable carrier. In some embodiments, the modified cells may be formulated using one or more excipients to, e.g.: (1) increase stability; (2) alter the biodistribution (e.g., target the cell line to specific tissues or cell types); (3) alter the release profile of an encoded therapeutic factor.

Formulations of the present disclosure can include, without limitation, saline, liposomes, lipid nanoparticles, polymers, peptides, proteins, and combinations thereof. Formulations of the pharmaceutical compositions described herein may be prepared by any method known or hereafter developed in the art of pharmacology. As used herein the term “pharmaceutical composition” refers to compositions including at least one active ingredient (e.g., a modified cell) and optionally one or more pharmaceutically acceptable excipients. Pharmaceutical compositions of the present disclosure may be sterile.

Relative amounts of the active ingredient (e.g., the modified cell), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may include between 0.1% and 99% (w/w) of the active ingredient. By way of example, the composition may include between 0.1% and 100%, e.g., between 0.5 and 50%, between 1-30%, between 5-80%, or at least 80% (w/w) active ingredient.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, MD, 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Injectable formulations may be sterilized, for example, by filtration through a bacterial-retaining filter, and/or by incorporating sterilizing agents in the form of sterile solid compositions which can be dissolved or dispersed in sterile water or other sterile injectable medium prior to use.

### EXAMPLES

The following example is offered to illustrate, but not to limit, the claimed invention.

### Example 1: Striated Muscle-Specific Base Editing Enables Correction of Mutations Causing Dilated Cardiomyopathy

### Abstract

Dilated cardiomyopathy is the second most common cause for heart failure with no cure except a high-risk heart transplantation. Approximately 30% of patients harbor heritable mutations which are amenable to CRISPR-based gene therapy. However, challenges related to delivery of the editing complex and off-target concerns hamper the broad applicability of CRISPR agents in the heart. We employ a combination of the viral vector AAVMYO with superior targeting specificity of heart muscle tissue and CRISPR base editors to repair patient mutations in the cardiac splice factor Rbm20, which cause aggressive dilated cardiomyopathy. Using optimized conditions, we repair >70% of cardiomyocytes in two Rbm20 knock-in mouse models that we have generated to serve as an in vivo platform of our editing strategy. Treatment of juvenile mice restores the localization defect of RBM20 in 75% of cells and splicing of RBM20 targets including TTN. Three months after injection, cardiac dilation and ejection fraction reach wild-type levels. Single-nuclei RNA sequencing uncovers restoration of the transcriptional profile across all major cardiac cell types and whole-genome sequencing reveals no evidence for aberrant off-target editing. Our study highlights the potential of base editors combined with AAVMYO to achieve gene repair for treatment of hereditary cardiac diseases.

### Introduction

Next-generation CRISPR tools enable gene repair of disease-associated mutations in situ, in the organ of interest, thereby achieving complete prevention or cure of the disease¹. To date, a few clinical trials have been initiated applying CRISPR in vivo to treat mutations causing blindness, high cholesterol, or protein aggregation^{2,3}. Hundreds of pathogenic single-nucleotide variants (SNVs) have been associated with cardiac diseases, making the heart an attractive target for gene therapy⁴. However, few attempts have been made to correct heritable cardiac disorders in vivo. Several studies have corrected pathogenic cardiac mutations in mouse^{5,6,7} and human embryos⁸, which has major ethical considerations, and also requires prior knowledge of the inherited mutation. Others have disrupted exons by Cas9-mediated non-homologous end joining in mice⁹, dogs¹⁰ and pigs¹¹ entailing the danger for erroneous DNA repair, which could impair gene expression.

Cardiomyocytes are non-proliferating cells that are impervious to homology-directed gene repair, the method of choice for installing precise genome edits. Recently, CRISPR base editors have been developed to allow efficient nucleotide conversions in vivo in post-mitotic cells¹. Thus, we evaluated the use of base editors for the treatment of familial dilated cardiomyopathy (DCM), a severe form of heart disease and the second most common cause of heart failure¹². Treatment options for DCM patients include drugs to reduce blood pressure or block the neurohormonal system. However, with a 15-year survival rate of only 34%¹³, the mortality of DCM patients receiving this treatment is very high¹⁴. We focused on mutations in RBM20, found in 3% of patients with aggressive, early onset DCM15. Patients with familial RBM20-DCM normally harbor a single-nucleotide disease-causing variant¹⁶ making it a prime target for base editors to install single-nucleotide conversions. RBM20 encodes a cardiac splice factor that regulates alternative splicing of genes critical for the function of cardiomyocytes¹⁶. RBM20 mutations are enriched in a small stretch of six amino acids within the RS-domain and were recently shown to result in aberrant formation of cytoplasmic granules, which likely amplify the disease phenotype^{17,18,19}.

Besides correcting the mutation, the major goal for any CRISPR-related gene therapy is to attain organ-specific gene delivery to reduce the chance of potentially deleterious off-target editing. Due to their low risk of immunogenicity²⁰ and integration²¹, as well as their high amenability to genetic retargeting to desired organs, adeno-associated viruses (AAVs) present one of the safest and most versatile options for gene delivery. Previous cardiac gene transfer has been performed with the serotype AAV9 despite its predominant targeting of the liver upon intravenous injection²². We have recently identified a synthetic variant of AAV9, named AAVMYO, which exhibits high target affinity for muscle cells including cardiomyocytes and low affinity for other organs such as the liver²². Here, we leverage AAVMYO for systemic delivery of base editors to cardiomyocytes, the main cell type expressing Rbm20. We optimize the strategy to selectively repair two pathogenic mutations in Rbm20's RS-domain resulting in near-complete prevention of the disease phenotype in mice, with no evidence for guide RNA (gRNA)-dependent off-target activity.

### Results

### P635L and R636Q Rbm20 Knock-In Mice Exhibit a DCM Phenotype

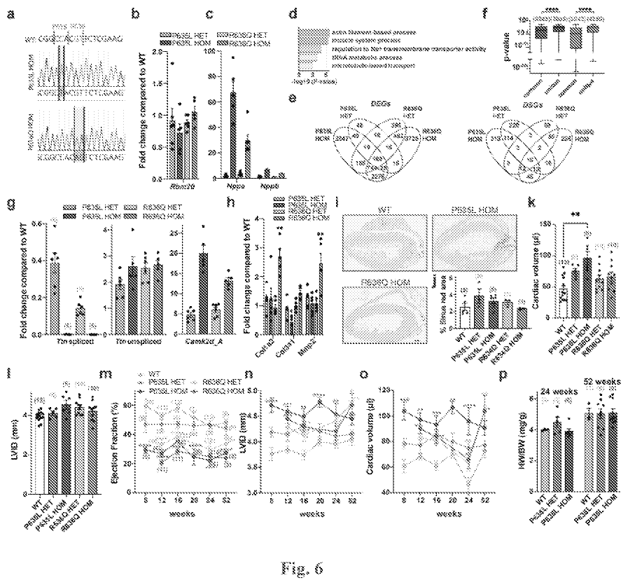

Adenine base editors (ABEs) convert adenines (A) to guanines (G) and have been used successfully in emerging clinical trials²³. Since none of the existing Rbm20 animal models are amenable to ABE-mediated nucleotide conversion, we generated two mouse models harboring G>A mutations. Specifically, we established two Rbm20 knock-in mouse models with the amino acid substitutions P635L and R636Q, respectively, orthologous to the RBM20 mutations P633L and R634Q in humans previously identified in DCM patients (FIG. 6a)²⁴. No significant changes were observed in Rbm20 mRNA expression in these mice (FIG. 6b). We performed deep phenotyping to identify aberrant molecular signatures and physiological traits that could be rescued upon base editing. We focused on RBM20 localization, gene expression and heart function since these parameters are dysregulated in mice, humans and pigs with RBM20 RS-domain mutations^{17,18,19}.

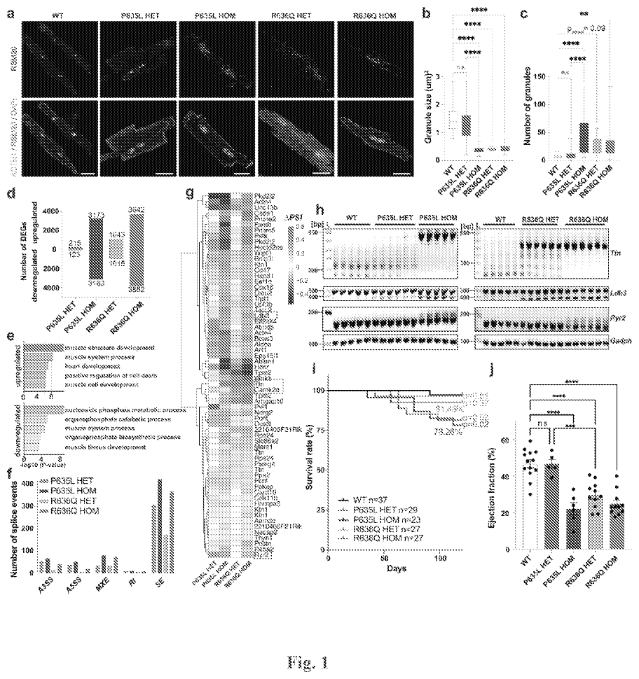

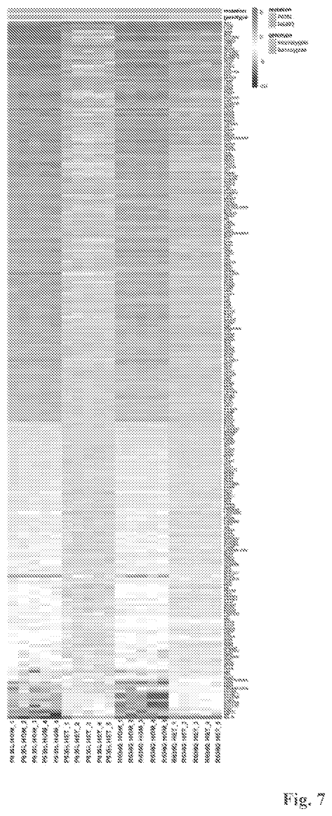

Immunostaining of isolated cardiomyocytes showed that homozygous (HOM) P635L and R636Q mutant mice have cytoplasmic RBM20 granules, indicating mislocalization of the mutant RBM20 protein from its normal nuclear localization (FIG. 1a). The heterozygous (HET) mutants diverged strongly in the degree of RBM20 mislocalization. While RBM20 was predominantly nuclear in P635L HET, it formed small cytoplasmic granules in R636Q HET mice (FIG. 1a-c). RNA-sequencing (RNA-seq) revealed that the number of differentially expressed genes (DEGs) compared to wild-type (WT) was sixfold higher in R636Q HET compared to P635L HET but lower than in P635L and R636Q HOM mice (FIG. 1d). DEGs common for both P635L and R636Q exhibited dose-dependency between HET and HOM (FIG. 7). Gene ontology (GO) analysis of the common DEGs revealed dysregulation of genes involved in muscle function and metabolic genes (FIG. 1e). The expression of natriuretic peptide precursors A and B (Nppa and Nppb), which are biomarkers of heart failure²⁵, was substantially elevated in HOM mice (FIG. 6c). We identified 58 differentially spliced genes (DSGs) common for both P635L and R636Q HOM mice with the predominant splice event being exon skipping (FIG. 1f). These DSGs were associated with muscle and cytoskeletal functions (FIG. 6d). Notably, approximately half of all DEGs and DSGs did not overlap between P635L and R636Q HOM (FIG. 6e). While these specific genes could suggest the presence of mutation-specific downstream processes, their P values were higher on average than for overlapping genes (FIG. 6f). This is consistent with the detection of subtle changes in transcript abundance arising due to biological variation, such as between mice, or other confounding factors that were detected by our deep RNA-seq with 100 Mio. reads on average per genotype. While no difference in the abundance of splice events between P635L and R636Q HET was observed (FIG. 1f), a subset of crucial RBM20 targets including Tin, Camk2d and Tpm2 were more dysregulated in R636Q HET compared to P635L HET (FIG. 1g). We performed RT-PCR and qPCR to validate the isoforms of Ttn, Camk2d, Ryr2 and Ldb3, which were differentially expressed in mutant mice, and observed stronger dysregulation of Tin and Ldb3 in R636Q HET compared to P635L HET mice (FIG. 1h and FIG. 6g). P635L and R636Q HOM exhibited similar levels of aberrant splicing and stronger than the HET mice (FIG. 1g, h and FIG. 6g).

Next, we investigated whether these molecular differences in both mouse models affected the cardiac phenotype. Survival curves indicated that both P635L and R636Q HOM mice died prematurely in the first 120 days albeit to a lesser extent than other Rbm20 RS-domain mutations (survival rate: 78% P635L, 81% R636Q, 66% S637A²⁶, 51% S639G²⁷) (FIG. 1i). Notably, we backcrossed our mutant mice to C57BL/6J where others have used C57BL/6N²⁸, which could explain the differences in the survival as C57BL/6N is more susceptible to cardiac deterioration upon pressure overload²⁹. Both histological analysis and gene expression did not uncover major signs of fibrosis in 16-week-old mutant mice except upregulation of the fibrosis marker Colla2 and Mmp2 in R636Q HOM mice (FIG. 6h-j). The clinical definition of DCM is based on an ejection fraction of <45% and left ventricular dilation¹². We performed narcosis echocardiography, which confirmed that both mouse models exhibit a DCM phenotype with significantly reduced ejection fraction (FIG. 1j). However, they displayed only minor increase in cardiac volume (except in P635L HOM) and no significant change in the left ventricular internal diameter (LVID) (FIG. 6k, l). Corroborating the RNA-seq results, and correlating with cytoplasmic granule formation, the ejection fraction was more reduced in R636Q HET compared to P635L HET mice. After 1 year, no significant worsening of the DCM-associated phenotype was observed in ejection fraction and cardiac volume for P635L HET and HOM mice whereas LVID and cardiac volume significantly increased in R636Q HET and HOM mice (FIG. 6m-p). All mutant mice exhibited consistently higher LVID and cardiac volume compared to WT. We conclude that P635L and R636Q Rbm20 mutant mice exhibit DCM characteristics found in animals and patients with other RS-domain mutations^{17,18,19}. For subsequent rescue strategies, we focused on P635L and R636Q HOM mice since they showed a more pronounced molecular and physiological defect enabling better quantification of the efficacy of the base editor treatment.

### Base Editors Repair Pathogenic Rbm20 Mutations In Vitro and in Mice

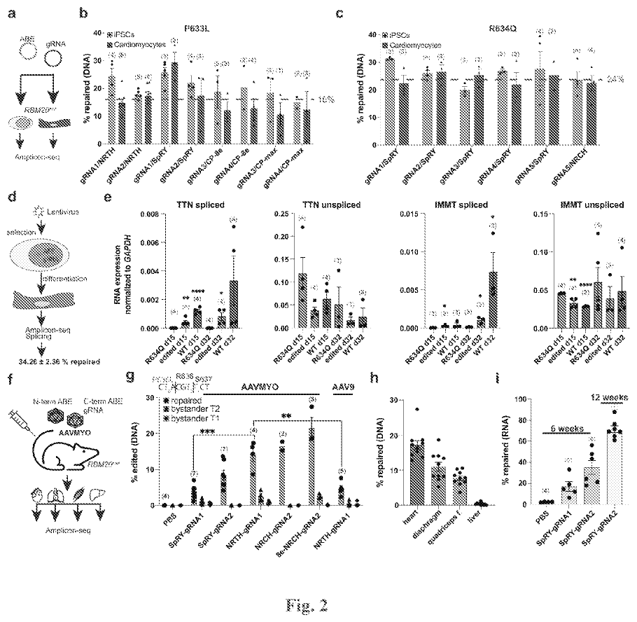

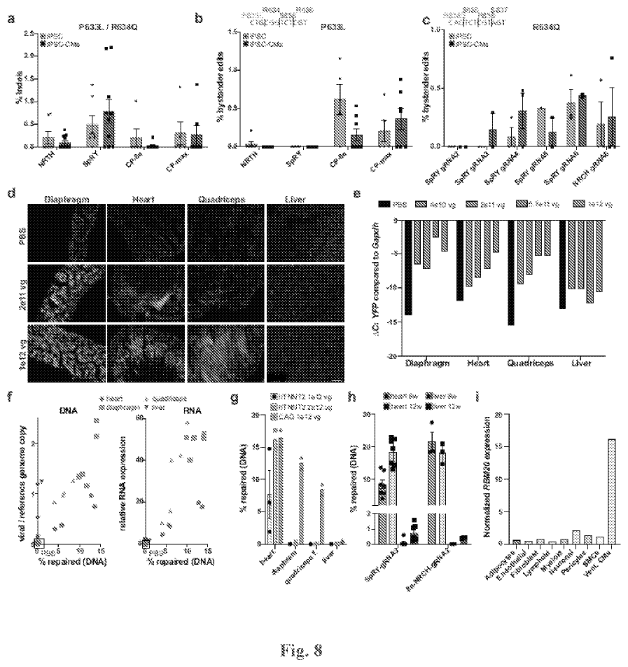

To test the feasibility of base editor treatment for repairing pathogenic P635L and R636Q mutations, we first transfected ABEs combined with compatible gRNAs in proliferating human iPSCs and non-proliferating cardiomyocytes derived from induced pluripotent stem cells (iPSC-CMs) with the orthologous RBM20 mutations P633L and R634Q (FIG. 2a). Due to sequence restrictions, we used ABEs containing Cas9 that recognize non-canonical PAMs such as “NRN” used in conjunction with the ABE SpRY30, or the ABEs NRTH/NRCH named after their PAM preference³¹. Moreover, we tested circular permuted ABE (CP-1041) exhibiting a broader editing window³² for targeting of canonical PAMs in P633L. We observed comparable editing efficiencies of RBM20 mutations between iPSCs and iPSC-CMs of up to 30% on average (FIG. 2b, c). No base editor clearly outperformed others. Indel formation, a byproduct of base editors^{30,31}, was below 2.5% with no significant bias between different ABEs (FIG. 8a). Likewise, bystander edits (i.e., unwanted A>G conversions within the gRNA window) were generally below 1% with no significant trend between different base editors except for circular permuted editors, which led to more bystander edits for P633L likely due to their broader editing window (FIG. 8b, c). Next, we analyzed whether editing of iPSC-CMs restored RBM20-mediated splicing. We generated R634Q iPSCs with stable expression of the base editor SpRY together with a targeting gRNA using lentiviral transduction leading to a repair efficiency of 34% (FIG. 2d). After differentiation to iPSC-CMs, the expression levels of spliced isoforms of TIN and IMMT, prominent RNA targets of RBM20³³, were increased in expression whereas the unspliced isoforms were decreased in base-edited cells suggesting that base editing restored RBM20-related splice defects (FIG. 2e).

Encouraged by these results, we tested the performance of base editors in editing of the heart in vivo. We utilized a split-intein strategy³⁴ to package both parts of the ABE (controlled by the constitutive CAG promoter) together with a gRNA expression cassette into the synthetic AAV9 variant AAVMYO (referred to as AAVMYO-ABE) (FIG. 2f). To determine optimal virus concentration for systemic delivery, we used a YFP reporter transgene and observed that 1e12 vector genomes (vg) (corresponds to 8.33e13 vg/kg total virus concentration) ensures high viral targeting of the heart without overt expression of the transgene in the liver (FIG. 8d, e). To identify the optimal base editor-gRNA combination, we tested the in vivo editing performance of the ABEs NRTH, NRCH and SpRY. We performed tail vein injections of AAVMYO-ABEs combined with two different gRNAs in P635L HOM mice and analyzed editing of the mutation in the heart, diaphragm, quadriceps and liver after 6 weeks. Experiments were performed in juvenile 4-week-old mice resembling young DCM patients with the possibility to prevent disease progression. We found clear performance differences between both tested gRNAs. gRNA2 displayed higher on-target editing efficiency than gRNA1, and fewer bystander edits (FIG. 2g). The base editors NRCH and NRTH outperformed SpRY with regards to editing efficiency (FIG. 2g), in contrast to in vitro editing where no clear differences between different ABEs were observed. We also tested NRCH conjugated with the latest and most efficient version of adenine deaminase, namely Abe8e³⁵ (referred to as 8e-NRCH), and observed the highest editing with 21.4% on average (FIG. 2g). This editor, however, also showed bystander edits of 2.7%, the most common bystander edit of which (T2; 2.64%) introduces a synonymous codon change and is likely inconsequential. Of note, due to different positioning of the base editor, a second non-synonymous bystander edit (T₁) was observed for gRNA1 in up to 1.31% of reads leading to a codon change from TCT (serine) to CCT (proline). Therefore, the use of gRNA1 was discontinued for subsequent long-term editing and phenotyping. T₁ was also detected in 8e-NRCH combined with gRNA1 but only in 0.09% of reads on average. No indels were observed in any condition. For NRTH, we also generated AAV9 vectors, which exhibited less than half of the editing efficacy of the AAVMYO-ABE counterpart supporting the superiority of AAVMYO for cardiac gene delivery (FIG. 2g). Notably, no significant editing was observed in the liver. Highest editing occurred in the heart followed by diaphragm and quadriceps suggesting that the liver and likely other non-muscle tissue are protected from on-target but also off-target base editing activity (FIG. 2h). Viral DNA copy number and relative RNA expression correlated broadly with editing efficiency (FIG. 8f). Since AAVMYO predominantly infects cardiomyocytes, which constitute only 30-50% of all cardiac cells^{36,37}, the viral expression measured in the heart is likely an underestimate.

We also generated an ABE version driven by the promoter of human cardiac troponin T (hTNNT2), which led to highly specific editing in the heart and absence of editing in other tissues (FIG. 8g). However, hTNNT2-driven ABEs were only comparable in editing efficacy with constitutive CAG promoter-driven ABEs when doubling the virus concentration. Since we sought to exclude potential side effects from high viral loads in the mouse model, we continued mainly with CAG-driven ABEs. To evaluate the potential of long-term base-editing, we collected the heart 12 weeks instead of 6 weeks after injection. Whereas 8e-NRCH outperformed SpRY after 6 weeks, both exhibited similar levels of editing after 12 weeks (FIG. 8h). Editing of the liver did not exceed 2% even 12 weeks after injection (FIG. 8h).

Finally, we quantified the extent of Rbm20 mRNA that was edited, since this allows estimating the editing efficacy in cardiomyocytes, i.e., the cell type that predominantly expresses Rbm20 (FIG. 8i) 37. Sequencing of heart cDNA after 6 weeks of editing revealed that on average 35% of mRNA molecules were edited with SpRY compared to 8% on the DNA level (FIG. 2g, i). Strikingly, 12 weeks after editing, 71% of Rbm20 mRNA were edited on average compared to 18% of DNA (FIG. 2g, i). Similar to the copy number measurements, the discrepancy between the extent of DNA and RNA editing is likely due to the fact that AAVMYO only infects cardiomyocytes, which overall represent a smaller fraction of the DNA extracted from the heart. We conclude that base editors delivered with AAVMYO enable highly efficient muscle-specific repair of Rbm20 mutations in mice.

### Base Editors Repair Rbm20-DCM Phenotypes in Mice

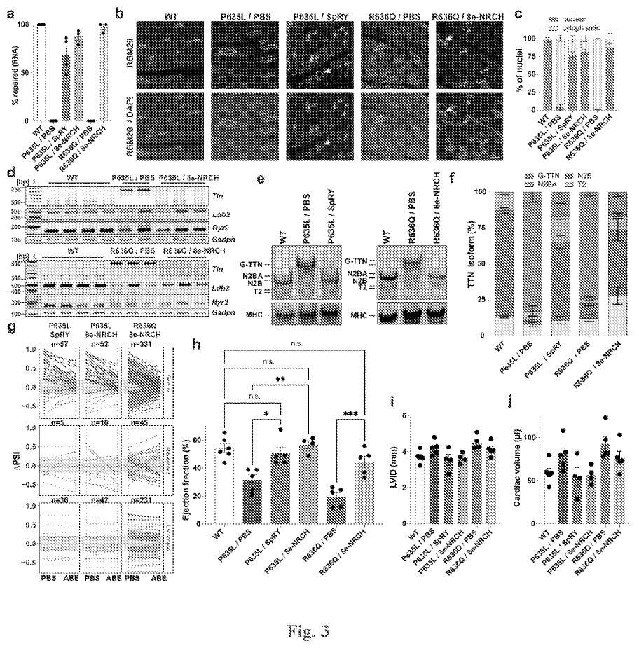

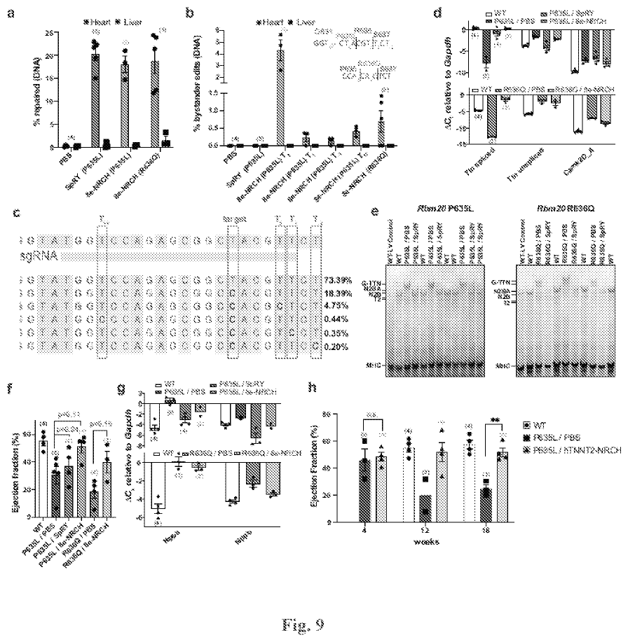

To measure long-term effects, we performed tail vein injections of AAVMYO-ABE, using our best performing editor-gRNA combinations 8e-NRCH and SpRY, the latter exhibiting less bystander edits. These injections were performed in 4-week-old P635L and R636Q HOM mice. Long-term base-editing and physiological effects were analyzed 12 weeks after injection. Amplicon-seq of whole heart gDNA revealed an average repair efficiency of 18-20% in the heart and below 2% in the liver across the two tested Rbm20 mutations (FIG. 9a). As observed before with gRNA2, bystander edits were detected in mice treated with 8e-NRCH but not in mice injected with SpRY (FIG. 9b). Compared to editing after 6 weeks, overall, more bystander edits were detected in P635L mice treated with 8e-NRCH. The main synonymous bystander edit T₂ occurred in 4.09% of reads on average followed by a missense mutation T₁ (0.33%). Two other synonymous mutations T₋₂ (0.20%) and T₁₇ (0.42%) were observed (FIG. 9b). R636Q mice treated with 8e-NRCH exhibited one bystander edit A2 (0.69%). Notably, we observed bystander edits only in reads that have also received the correct edit indicating that only repaired alleles were prone to bystander edits, which effectively lowers the editing efficacy (FIG. 9c). Indels surrounding the gRNA window were not detectable. Also, levels of Rbm20 mRNA editing were substantially higher, namely 68% for SpRY and >85% for 8e-NRCH (FIG. 3a).

To evaluate the extent of phenotype rescue upon base editing, we performed RBM20 localization and gene expression assays, as well as analysis of heart pump function. RBM20 immunostaining in heart tissue sections revealed eradication of the cytoplasmic RBM20 granules and restoration of the characteristic nuclear RBM20 foci in 75% of cells in AAVMYO-ABE-treated mice compared to control mice injected with saline (FIG. 3b, c). Next, we analyzed splicing of Tin and observed increased expression of the spliced, as well as decreased expression of the unspliced isoforms. Moreover, the splicing profile of other RBM20 targets Camk2d, Ldb3 and Ryr2 approached levels of the WT control (FIG. 3d and FIG. 9d). Since Tin mis-splicing likely contributes to aberrant cardiomyocyte function in RBM20-DCM38, we validated TTN expression at the protein level. AAVMYO-ABE-treated mice showed reduced expression of the gigantic TTN isoform (G-TTN) from 83 to 17%, with levels of constitutive N2A and N2BA isoforms approaching levels of WT (FIG. 3e, f and FIG. 9e). RNA-seq of PBS or ABE-treated P635L and R636Q HOM mice revealed that about 50% of the mis-spliced exons in PBS-treated mice were rescued after base editing; especially Tin exons were amongst the most strongly reverted splice events (FIG. 3g).

Finally, we performed narcosis echocardiography 8 and 12 weeks after injection. After 8 weeks, there was a clear but not significant trend toward an increase in the ejection fraction (FIG. 9f). However, after 12 weeks, the ejection fraction was reverted almost to WT levels (FIG. 3h). In line with the restoration of cardiac function, LVID and cardiac volume decreased upon base editing albeit without reaching statistical significance (FIG. 3i, j). Moreover, expression of the heart failure biomarkers Nppa and Nppb was reduced after base editing compared to PBS-injected samples (FIG. 9g). Notably, we also performed echocardiography after injection of hTNNT2-driven ABE and observed significant improvement of the ejection fraction after 12 weeks (FIG. 9h). We conclude that AAVMYO-ABE delivery significantly improves the molecular and physiological defects associated with Rbm20 mutations in mice.

### Rescue of Cell Type-Specific Gene Expression Upon Base Editing

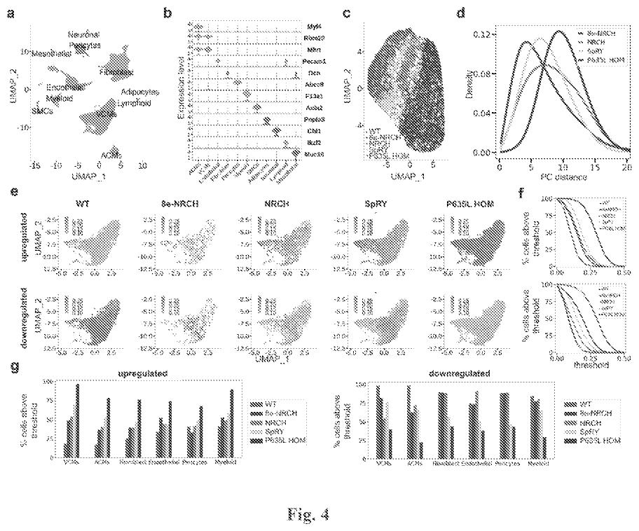

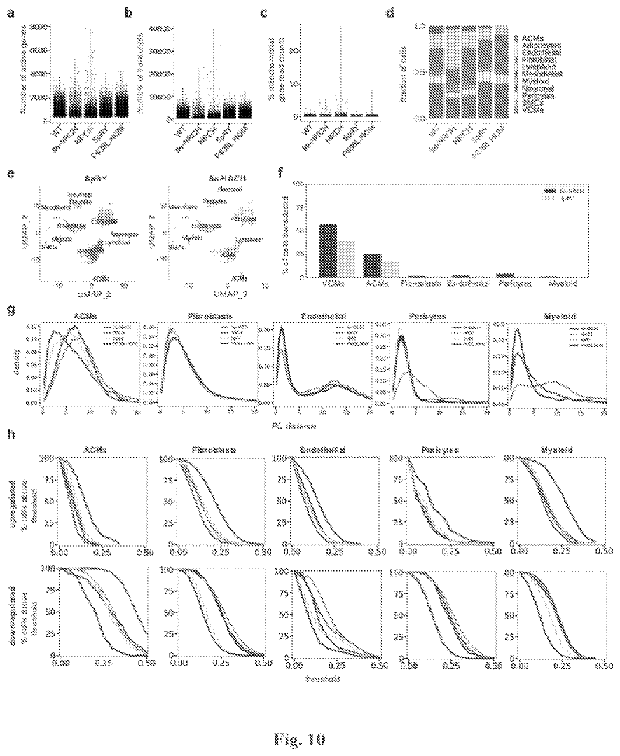

To investigate whether base editing restores the transcriptional landscape of the heart, we performed single-nuclei RNA sequencing (snRNA-seq) of 40,235 nuclei isolated from hearts of 16-week-old mice in the absence and with base editor treatment. We analyzed nuclei from WT (n=7867), P635L HOM (n=16,218), and P635L HOM mouse hearts 12 weeks after injection of AAVMYO with NRCH (n=6246), 8c-NRCH (n=2286) or SpRY (n=7618) (FIG. 10a-c). UMAP projection based on transcriptional similarity and clustering identified 11 major cell types that express known cell type markers found in previous studies³⁷ (FIG. 4a, b). Sub-clustering within the ventricular cardiomyocytes revealed that cells from base-edited mice have transcriptional profiles between those of WT and P635L HOM mice (FIG. 4c). The fraction of immune cells (lymphoid and myeloid) increased only slightly upon AAVMYO treatment (2.4-4% in WT and P635L HOM, 3.6-5.6% in base-edited mice) indicating the absence of an overt immune response (FIG. 10d). In our snRNA-seq data, we also analyzed the expression of the base editor complex itself and confirmed predominant targeting of cardiomyocytes by AAVMYO (FIG. 10e, f). Next, we compared transcriptome similarities between WT, P635L HOM and P635L HOM after base editor treatment. In ventricular cardiomyocytes, cells after base editor treatment shifted closer to WT in their transcriptional profile whereas no overt trend was observed for the other major cell types (FIG. 4d and FIG. 10g). Since transcriptome effects could be masked by genes unrelated to the Rbm20 mutation, we analyzed the transcriptomic profile for genes that were significantly dysregulated in P635L HOM mice (based on snRNA-seq, see ‘Methods’ section snRNA-seq analysis). Ventricular cardiomyocytes from base-edited mice exhibited a gene expression profile that is between WT and P635L HOM, indicating that gene expression was at least partially restored (FIG. 4e-g). Strikingly, we also observed major gene expression changes in other cell types with levels reaching WT levels for atrial cardiomyocytes, pericytes, endothelial cells, myeloid cells and fibroblasts (FIG. 4g and FIG. 10h). This indicates that downstream effects associated with RBM20-DCM such as changing the gene expression profile of non-cardiomyocytes were repaired even though the AAVMYO-ABE treatment specifically targets cardiomyocytes.

### No Evidence for AAVMYO-ABE-Induced Off-Target Editing

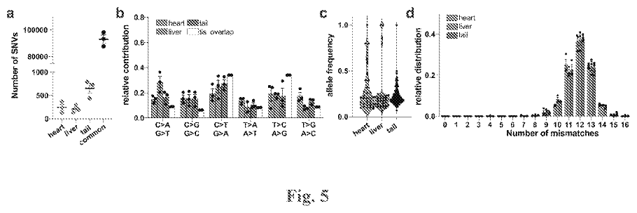

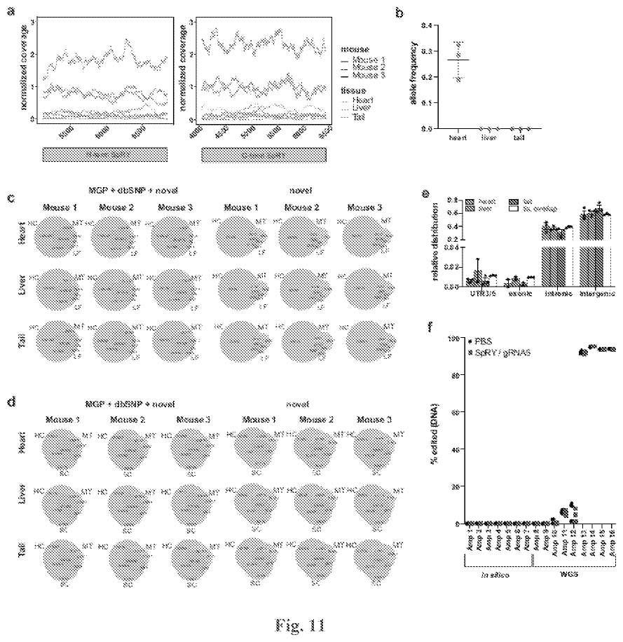

Finally, we sought to identify off-target mutations induced by the base editor. We performed whole-genome sequencing (WGS) in three P635L HOM mice treated with AAVMYO and the base editor SpRY for 12 weeks. For each mouse, we sequenced tail (harvested before the injection), liver and heart tissue with an average genome coverage of 47×. WGS confirmed a high viral load in the heart, low levels in the liver and background signal in the tail (FIG. 11a), with an average on-target allele editing frequency of 27% in the heart and absence of editing in the other tissues (FIG. 11b). We adapted a previous strategy³⁹ (see ‘Methods’ section whole-genome sequencing and analysis) to identify novel variants for each tissue by overlapping three variant callers that identify SNVs and indels (FIG. 11c, d). We focused on variants that were detected by at least two variant callers. After applying additional filter steps, we found on average 208-650 tissue-specific variants in the heart, liver, tail (FIG. 5a). The relative contribution of A>G/T>C nucleotide conversions was not increased in the heart compared to other tissue-specific variants or variants that overlap in all three tissues (FIG. 5b). Moreover, the allele frequency of A>G/T>C mutations was similar in all three tissues (FIG. 5c), indicating the absence of systematic off-target mutations installed by this ABE. The genomic distribution of tissue-specific variants was similar to common variants with only a small fraction of SNVs in exonic regions (FIG. 11e). None of the heart-specific variants were shared between the three replicates and seven were identified in two mice. Only one SNV was found to change the amino acid sequence of a coding gene. No sequence homology was detected in the genomic region surrounding the novel variants compared to the gRNA sequence, which suggests the absence of gRNA-dependent editing (FIG. 5d). We further analyzed 16 selected sites by amplicon-seq: 7 loci with highest sequence similarity to the gRNA used and 9 candidate A/T>G/C variants determined by WGS with the highest sequencing coverage (FIG. 11f). No SNVs were detected in the in silico predicted off-target sites. In addition, 4 out of 9 candidate loci from WGS were >90% mutated in both PBS and AAVMYO-ABE-treated mice and therefore are likely germline variants. For the remaining 5 SNVs, no difference in the percentage of editing was observed after AAVMYO-ABE injection compared to PBS. Overall, this data does not indicate the presence of ABE-induced DNA off-target edits.

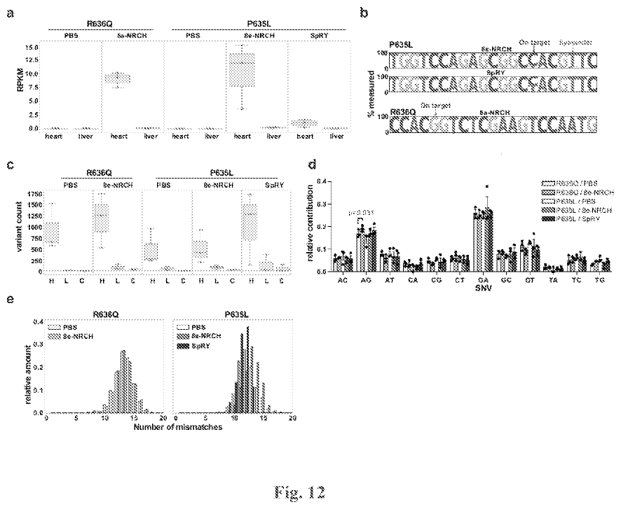

Since RNA editing has been reported as byproducts of ABEs^{40,41}, we also analyzed bulk RNA-seq data obtained 12 weeks after AAVMYO-ABE treatment of P635L and R636Q HOM mice. We confirmed high expression of the base editor in the heart and its absence in the liver (FIG. 12a) leading to high on-target and minor bystander editing for 8e-NRCH in P635L HOM (FIG. 12b). Unbiased variant detection was performed on the RNA-Seq data (FIG. 12c; details in the method) and a small but significant increase (from 17% to 19%) in the fraction of A>G mutations was observed only in the 8e-NRCH compared to PBS-treated R636Q HOM mice (FIG. 12d). No significant differences were observed in the other AAVMYO-ABE-treated samples. Similar to WGS, we could not detect major sequence homology between the region surrounding the variants and the gRNA (FIG. 12e) indicating that the increased frequency of A>G mutations is not due gRNA-dependent effects. In summary, while our analysis prohibits the detection of random SNVs arising in a subset of cells, we do not find evidence that the base editing strategy induces systemic off-target editing.

### Discussion

Chances of success of any gene repair strategy depend on the degree of editing reached in the target cell type. This efficiency is contingent on the extent of editor delivery in the target cell type and its gene editing efficacy within each cell. Since Rbm20 is mainly expressed in cardiomyocytes, we could infer overall editing efficiency by analyzing Rbm20 mRNA. Strikingly, 70-87% of cardiomyocytes were repaired, demonstrating both the superior targeting capability of AAVMYO and the efficacy of base editors, specifically ABEs. In a recent study, Nishiyama and colleagues employed base editors delivered by AAV9 in 5-day-old mice and achieved an Rbm20 mRNA editing efficiency of 66% on average²⁸. However, with a total concentration of 2.5e14 vg/kg, 3-times more virus was used than in our study where we treated larger, juvenile 4-week old mice. The differences in AAV amount can be attributed to promoter choice and AAV serotype. Nishiyama et al. employed a TNNT2 (cTnT) promoter to confer cardiac-specific base editor expression, which in our hands required twice the AAV amount to reach editing efficiencies similar to expression controlled by the constitutive CAG promoter. Using a similar strategy but combined with thoracic injections, another study recently achieved 81% editing efficiency of cDNA in the left ventricle⁴². Notably, since AAVMYO selectively targets cardiomyocytes and not the liver, less virus is required for cardiac delivery compared to AAV9. In our hands, base editors delivered by AAV9 were only half as efficient in correcting Rbm20 mutations compared to AAVMYO-ABE when injected in the same concentrations. The advantages of this higher targeting specificity are two-fold. First, high AAV concentrations are associated with toxicity due to AAV-induced adaptive immune response⁴³. Second, AAV production is a time-consuming and costly process. Especially for human therapies, up to 1.5e17 vg of virus have been applied⁴⁴ and using a more efficient AAV helps to reduce the required virus concentration. Moreover, it allows the treatment of older (and heavier) specimen, thereby enabling therapies in adults which is usually the stage when subjects develop first symptoms or genetic tests are initiated⁴⁵.

Off targets represent a major concern for in vivo gene therapy in patients. To uncover potential off-target editing associated with CRISPR-treatment, WGS is the method of choice because it offers an unbiased analysis of SNVs across the genome in vivo³⁹. We showed that after treatment, A>G/T>C nucleotide conversions were not enriched in the hearts that received a high dose of AAVMYO-ABE. This argues against rogue base editing of DNA elsewhere in the genome as it was observed in earlier base editor versions, especially for cytosine base editors⁴⁶. In addition, we did not identify sequences similar to the gRNA near heart-specific SNVs, which indicates that detected SNVs are not related to guide directed activity. Notably, we observed only one missense mutation out of 768 heart-specific SNVs. However, since WGS suffers from sensitivity, it remains unknown whether base editors installed random mutations of low frequency in a subset of cells. Uncovering such edits would require clonal amplification of the target cell type prior to WGS. While such a strategy has been performed for hepatocytes⁴⁷, it would not work for cardiomyocytes due to their inability to proliferate. At RNA level, we showed a significant increase from 17 to 19% of A>G edits in one mouse strain treated with 8e-NRCH, which could indicate rogue off-target editing of the base editor. Since we observed this effect only for R636Q HOM mice treated with 8e-NRCH without overt gRNA-dependent effects, the risk for introducing permanent changes in gene expression is low. Besides off-target edits, base editors are prone to bystander edits in the vicinity of the targeting window¹. The percentage of bystander edits varied dependent on the deaminase. SpRY and NRCH conjugated to Abemax did not lead to bystander edits whereas NRCH conjugated to hyperactive Abe&e deaminase led to substantial bystander edits of >4% even outside of the putative editing window. Dependent on the target mutation it might make sense to utilize the less active Abemax-ABE version to reduce the danger of bystander edits. In summary, due to the low abundance of random DNA and RNA off-target edits and the reduction of bystander edits by choosing the best ABE, one could argue that the benefit of dramatically decreasing the risk of heart failure outweighs the danger of detrimental off-target editing.

We showed that the extent of Rbm20 mRNA correction matched well with the number of cardiomyocyte nuclei exhibiting re-localization of RBM20 protein (75%) and the percentage of G-TTN reduction (from 83 to 17%) demonstrating molecular restoration of cardiomyocytes. In addition, we also observed a transcriptional shift towards wild-type in other cardiac cell types such as fibroblasts and epithelial cells upon base editing. SnRNA-seq in RBM20 patients showed major changes in gene expression and abundance of other cell types besides cardiomyocytes⁴⁸, therefore it is encouraging that non-cardiomyocytes also benefitted from the treatment. Overall, our data suggest that base editors prevent the permanent deterioration of heart function. Therefore, we speculate that the positive effect on all cell types is due to lack of structural changes of the heart occurring throughout the live span of the animal. Besides constituting a preventative action for genetically predisposed carriers that have not yet developed DCM, the AAVMYO-ABE treatment may be employed as a curative strategy of adult patients with DCM symptoms.

### Methods

### Mouse Line Generation

Rbm20-P635L and Rbm20-R636Q knock-in mice were generated by zygotic microinjection of recombinant Cas9 (IDT), in vitro reconstituted crRNA:trcrRNA (IDT) targeted to Rbm20, and single-stranded donor DNA as a template. The hybrid mouse strain B6C3F1 was used and backcrossed to C57BL/6J for experiments.

### Cell Culture and Differentiation

Parental iPSCs and iPSCs harboring the homozygous P633L or R634Q mutation in RBM20 were previously generated and characterized²⁴. Cells were maintained on vitronectin (A31804, ThermoFisher) coated plates with Essential 8™ Flex (A2858501, ThermoFisher) medium and passaged with Versene (15040066, ThermoFisher). Cardiomyocyte differentiation was initiated by addition of 8 μM CHIR99021 (72054, STEMCELL Technologies) in RPMI-1640 medium supplemented with B27 without Insulin (RPMI-Insulin, A1895601, ThermoFisher). After 24 h, 1 volume of RPMI-Insulin was added and after 72 h, medium was changed to RPMI-Insulin with 2 μM Wnt-C59 (5148, Tocris). At day 5 and 7, medium was changed to RPMI-Insulin and at day 9 to RPMI with full B27 supplement (RPMI+Insulin, 17504044, ThermoFisher). At day 11, medium was changed to RPMI+Insulin without glucose and addition of 5 mM DL-lactate. At day 14, RPMI+Insulin was added and at day 16, cells were passaged with TrypLE10× (A1217701, ThermoFisher) and RPMI+Insulin supplemented with 10% knock-out serum replacement (10828028, ThermoFisher) and 1.66 μM Thiazovivin (72252, StemCell Technologies). One day after passaging, the medium was changed to RPMI+Insulin with subsequent medium exchange every 3 days. Passaging was done every 2-3 weeks.

### ABE Plasmid Cloning

For the transient transfection of base editors in human iPSCs and iPSC-CMs, the following plasmids were used: ABEmax-NRTH (Addgene ID: 136922), ABEmax-NRCH (Addgene ID: 136923), ABEmax-SpRY (Addgene ID: 140003), ABEmax-CP-1041 (Addgene ID: 119808) and ABE8e-CP-1041 (Addgene ID: 138493). Forward and reverse complementary gRNA sequences with compatible overhangs were annealed and ligated with a gRNA expression plasmid (Addgene ID: 53188), which was digested with BbsI (R0539S, NEB) prior to ligation. For stable base editor expression, the coding region of Cas9 from the lentiCRISPRv2 plasmid (Addgene: 52961) was replaced with SpRY following a similar strategy as described before⁴⁹. The resulting plasmid was digested with BsmBI (R0580S, NEB) and ligated with the annealed gRNA. For the generation of split-intein ABE plasmids, we used Cbh_v5 AAV-ABE N-terminal (Addgene: 137177) and Cbh_v5 AAV-ABE C-terminal (Addgene: 137178) and replaced the coding sequence of SpCas9 with the N- or C-terminal parts of Cas9-NRTH, Cas9-NRCH or Cas9-SpRY using the plasmids from above as template. Moreover, we created a Abe8e-NRCH version by replacing ABEmax on the N-terminal part (common for Cas9-NRTH and Cas9-NRCH) with AbeBe using Addgene plasmid 138489 as template. The C-terminal AAV plasmids were digested with BsmBI and ligated with the annealed oligonucleotides encoding the gRNA. Gibson assembly (E2611L, NEB) was used for all cloning assembly steps except for gRNA oligos, which were ligated with the backbone using T4-DNA ligase (M0202L, NEB). Sanger sequencing was performed to validate plasmid assembly and Smal (R0141S, NEB) digestion to monitor the integrity of the ITRs.

### Lentivirus Production

The lentivirus (generation 3) was produced in Lenti-X 293T cells (632180, Takara) by transfecting the four plasmids with linear PEI (polyethylenimine, 25 kD). Virus was collected after 72 h (stored at 4° C.), fresh medium was added and virus was collected again 48 h later. All harvested virus was filtered with 0.45 μm low protein binding/fast flow filter unit. Virus was precipitated using Lenti-X-concentrator (631232, Takara) following the manufacturer's recommendations. Virus was further concentrated by ultracentrifugation with a 20% sucrose cushion at 50,000×g for 2 h at 4° C., and was resuspended in sterile 1×HBSS. Titers were estimated with Lenti-X GoStix Plus (631280, Takara).

iPSC and iPSC-CM Base Editing

IPSCs and iPSC-CMs were dissociated one day prior to plasmid transfection as single cells with StemPro™ Accutase™ cell dissociation reagent (A1110501, ThermoFisher) and re-seeded together with RevitaCell™ Supplement (A2644501, ThermoFisher) in 24-well plates coated with vitronectin. After 24 h, cells were transfected with 375 ng base editor plasmid, 125 ng U6 gRNA plasmid and 100 ng pmax-GFP (Lonza). Lipofectamine™ 3000 of Lipofectamine™ Stem transfection reagent (L3000008 or STEM00008, ThermoFisher) were used for transfection according to the manufacturer's instructions. Medium was changed 1 and 3 days after transfection, and GFP-positive cells were sorted by flow cytometry and analyzed by amplicon sequencing after 25-35 days. Generation of stable SpRY-expressing R636Q iPSCs was achieved by transducing the cells with Lentivirus expressing SpRY and R636Q gRNA2. Cells underwent Puromycin (A1113802, ThermoFisher) selection with 2 μg/ml for 14 days before expansion and differentiation to cardiomyocytes. Amplicon sequencing to measure RBM20 editing efficacy was performed before start of the cardiomyocyte differentiation in three independent replicates.

### Recombinant AAV Production, Purification, and Quantification

Recombinant AAVMYO was produced as previously described⁵⁰. Briefly, HEK-293T cells (Stratagene/Agilent) plated on 150 mm dishes were transfected using the 3-plasmid system (pAdH—adenoviral helper function, pRep2cap9myo²² encoding rep and cap genes, and transgene plasmid) and PEI. Cells were harvested 3 days later, and viruses were extracted from the cells by four rounds of freeze-thawing. The cell lysates were treated for 1 h with Benzonase to remove non-encapsidated DNA. To remove cell debris, the samples were centrifuged at 4000×g and the supernatant was collected. The supernatant was loaded over four layers of iodixanol gradient solution (15, 25, 40 and 60%), followed by a centrifugation for 2.5 h at 183,400×g (in average) in a 70Ti rotor. Fractions were collected and those corresponding to the interface of 40 and 60% were pooled, buffer exchanged and concentrated. The viral genome concentration (including in mouse tissue) was determined by ddPCR in a QX200 Droplet Digital PCR System (BioRad), using Taqman primers/probe against the CMV enhancer, and the purity by silver staining of SDS-PAGE gels.

Recombinant AAV9 was produced in HEK-293T/17 cells (ATCC; CRL-11268) using the triple-transfection method (with linear PEI 25 kDa) in a Corning CellSTACK 5 (CS5). After 72 h, supernatant (600 ml) was collected and stored at 4° C. and 600 ml of fresh medium was added. After an additional 48 h, the first collection was added back to the CS5, and cells were lysed and DNA was degraded by adding Triton X-100 (final concentration of 1%) and 94 μl Benzonase (25-35 U/μl) for 1 h at 37° C. with 100 rpm shaking. The cell debris/virus mix was removed and the CS5 was washed with 200 ml PBS. The washing solution and the cell suspension was centrifuged at 4000× g for 20 min. The supernatant was filtered with a 0.45 μm PES filter and then concentrated to 30 ml using tangential flow filtration. The concentrated virus was then purified by an iodixanol gradient and the titer was determined by qPCR using primers within the CMV promoter.

### Mouse AAV Injections

Mice were injected with a mix of AAVs expressing the N-terminal and C-terminal base editor or a YFP reporter. Unless otherwise specified, 5e11 vg per AAV were injected in the tail vein of 4-week-old mice. Mice weighted on average 12 g, therefore the total virus concentration injected was 8.33e13 vg/kg. Mice were sacrificed after 6 or 12 weeks and organs collected for subsequent analysis.

### DNA Isolation and Amplicon Sequencing

DNA from human cells was isolated using the Monarch® Genomic DNA Purification Kit (T3010L, NEB) following the manufacturer's instructions and including the recommended RNaseA digestion step. For DNA isolation from mice, the tissue was immersed in 600 μl PBS in tubes containing metallic beads and then processed with a Fastprep homogenizer with two 30 s runs with maximum velocity. One-third of the homogenized tissue was used for DNA isolation with the Monarch® Genomic DNA Purification Kit. No additional Tissue Lysis buffer was added and samples were incubated with 10 μl of Proteinase K for 1 h.

Purified DNA was amplified with human- or mouse-specific primers covering the RBM20 RS-domain mutation hotspot with Nextera-compatible adapters using QS® Hot Start High-Fidelity 2× Master Mix (M0494L, NEB). One microliter of a 1:100 dilution was used for a second PCR attaching sample-specific index barcodes (Nextera XT Index Kit v2 Set A, FC-131-2001, Illumina). Libraries were pooled and cleaned up with 1×AMPure XP beads (A63881, Beckman Coulter) before sequencing with a MiSeq instrument using a 150 bp paired-end run (Illumina).

Demultiplexed amplicons were analyzed using Crispresso2⁵¹ to obtain the frequency of on-target editing and indels and bystander edits with the extended gRNA binding sequence.

RNA Isolation, RT-PCR, qPCR

RNA from human cells was isolated using the Monarch® Total RNA Miniprep Kit (T2010S, NEB) following the manufacturer's instructions and including the on-column DnaseI digestion. For RNA isolation from mice, 1 ml of TRIzol™ (15596026, ThermoFisher) was added to 200 μl of homogenized tissue (see DNA extraction) and processed using the Direct-zol RNA Miniprep Kit (R2052, Zymo Research) with on-column DnaseI digestion. For the heart, RNA was isolated from the left ventricle. 200-500 ng RNA was used as input for the reverse transcription with SuperScript™ IV (18090010, ThermoFisher). For amplicon-seq, RNA was additionally treated with ezDNase™ (11766051, ThermoFisher) prior to reverse transcription. RT-PCR or qPCR was performed with 1 μl of 1:2 diluted cDNA using the Q5® Hot Start High-Fidelity 2× Master Mix (M0494L, NEB) or the SYBRIM Green PCR Master Mix (4309155, ThermoFisher), respectively, with gene-specific primers. Delta Ct method using Gapdh was used for sample normalization after qPCR. To calculate the fold change, an additional normalization relative to the averaged wild-type RNA expression was performed. RNA copy numbers were determined by ddPCR (see AAV virus quantification) using Taqman primers for the WPRE element and Rpp30 (Biorad, assay ID: dMmuCPE5097025) as housekeeping gene.

### Bulk RNA Sequencing and Analysis

500 ng of RNA isolated from left ventricles was processed using the NEBNext® Ultra II Directional RNA Library Prep Kit for Illumina® (E7760L, NEB) with prior enrichment of mRNA by using Oligo dT beads from the NEBNext Poly (A) mRNA Magnetic Isolation Module (E7490L, NEB). After library preparation, the samples were multiplexed (five samples per lane) and sequenced with Illumina NextSeq 2000. For bulk RNA-seq in FIG. 3, samples were processed according to the Smart-seq2 library preparation protocol⁵².

Subsequent analysis was performed using a pipeline assembled with Snakemake⁵³ available at: https://github.com/FerreiraAM/dem_Igreads_mouse_bulkRNA. The alignment of the different samples was performed using STAR⁵⁴. The GENCODE mouse annotation version vM29 with the primary assembly GRCm39 genome was used. We created the indexes and then aligned the reads for each sample using the default options of the STAR aligner. Differential expression analysis was performed with DESeq²⁵⁵. P values in FIG. 1d, e, FIG. 6e, f, FIG. 7 containing differential gene expression analysis were derived from a one-sided Wald test with adjustments for multiple comparisons. We performed comparisons for each mutation associated with each experiment in R⁵⁶ using the count matrices that were created from the BAM files with the Rsubread R package⁵⁷. Log 2 fold change (log 2FC) per individual was computed for each mutation, using the average per gene from all WT samples of one experiment's mutation: log 2FC for gene A=log 2 (value of gene A/WT average for gene A). Metascape⁵⁸ was used for Gene Ontology analysis.

We used rMATS59 to detect differential alternative splicing events. Pair-wise comparisons were performed and results from the Junctions Counts (JC) files were analyzed in R. We identified splice junction events that were overlapping between different conditions and filtered for significant events. P values in FIG. 1f, g, FIG. 6d, e containing differential splice analysis were derived from a likely-hood ratio test. An event was considered significant if the False Discovery Rate (FDR) was below 0.01 and the average delta PSI-value (relative to WT) was higher than 0.1 or smaller than-0.1. In FIG. 3g, significant splice events were classified in three categories: rescued, mis-spliced or unchanged. We computed the average ΔPSI difference (relative to WT) between PBS- and ABE-treated mice and considered the absolute difference (ΔΔPSI). Events were classified as unchanged if the ΔΔPSI was smaller than 0.1. The remaining events were either classified as rescued or mis-spliced. In addition, we defined the PSI values of PBS-treated samples as the original value ΔPSI_original, the PSI values of base-edited samples as the edited value ΔPSI_edited and used the following criteria:

Rescued:
ΔPSI_original > 0 and ΔPSI_edited >= 0 or −0.2 <= ΔPSI_edited <= 0.2 and ΔPSI_original >
ΔPSI_edited.
ΔPSI_original < 0 and ΔPSI_edited <= 0 or −0.2 <= ΔPSI_edited <= 0.2 and ΔPSI_original <
ΔPSI_edited.
Mis-spliced:
ΔPSI_original > 0 and ΔPSI_edited >= 0 and ΔPSI_original < x_edited.
ΔPSI_original < 0 and ΔPSI_edited <= 0 and ΔPSI_original > x_edited.
ΔPSI_original < 0 and ΔPSI_edited >= 0.2.
ΔPSI_original > 0 and ΔPSI_edited <= −0.2.

### Mouse Cardiomyocyte Isolation

For immunostainings of RBM20 granules (FIG. 1a-c), we performed a Langendorff-free isolation of cardiomyocytes as described before⁶⁰. Briefly, the mouse was sacrificed, and the right ventricle was immediately flushed with 7 ml of EDTA-containing buffer. After clamping the ascending aorta, the heart was transferred to a petri dish containing EDTA buffer. Another 10 ml of EDTA buffer was injected in the left ventricle. After injecting 3 ml Perfusion buffer, the heart was transferred to a Petri dish containing collagenase. Subsequently, the left ventricle was injected with 50 ml Collagenase buffer, transferred to a plate with 3 ml Collagenase buffer and cut in small pieces. Overall, 5 ml of stop solution was added and cells were filter through a 100 μm cell strainer and then settled by gravity to enrich for cardiomyocytes. Two rounds of gravity settling were performed before plating the cells on laminin (5 μg/ml in PBS, 23017015, ThermoFisher) coated plates with addition of DMEM/F12 with GlutaMAX™ (10565018, ThermoFisher) and 10% FBS. After 2 h, cells were fixed with 4% paraformaldehyde (PFA, methanol-free, 28906, ThermoFisher) for 10 min at RT and stored in PBS for subsequent imaging.

### RBM20 Immunostaining and Granule Quantification

Immunostainings were performed either in isolated adult mouse cardiomyocytes or in tissue sections. Isolated and fixed cardiomyocytes were incubated with 0.5% Triton X-100 in PBS for 5 min before washing with PBS and adding blocking solution (2% BSA diluted in PBS) for 1 h at RT. Cells were incubated overnight in blocking solution containing anti-Rbm20 (PA5-58068, Invitrogen) and anti-sarcomeric alpha-actinin (ab9465, Abcam) both diluted 1:250. Subsequently, cells were washed three times with blocking solution before staining with secondary antibodies Alexa Fluor 488 goat anti-mouse IgG (A11001, Invitrogen) and Alexa Fluor 568 goat anti-rabbit IgG (A110011, Invitrogen) both diluted 1:1000. Incubation was performed for 1 h at RT before washing three times with blocking solution and mounting the slides in ProLong Gold antifade reagent with DAPI (D1306, Invitrogen). Images were obtained by the LSM 980 AIRY confocal microscope (Zeiss). RBM20 granule quantification in isolated cardiomyocytes was performed by using the ImageJ plugin AggreCount (v1 13)⁶¹, according to the published instructions. The number and the average size of granules per whole cell were used for the figures. At least 10 cells were analyzed for each of three mice per genotype.

For tissue staining, transverse 8 μm sections of heart samples were deparaffinized with xylene and rehydrated to water through decreasing ethanol concentrations. Slide sections were subjected to heat-induced antigen retrieval for 20 min in 10 mM Tris-EDTA pH 9 buffer. Thereafter, these sections were permeabilized with 0.3% Triton X-100, blocked in 5% donkey serum and incubated overnight at 4° C. with a rabbit anti-Rbm20 antibody (PA5-53068, Invitrogen) at 0.5 μg/ml. Immunofluorescent detection was done with a tyramide signal amplification using an anti-rabbit-HRP (12-348, Sigma), Biotinyl-tyramide (SML2135, Sigma) and streptavidin-Alexa 488 (S11223, Molecular Probes). Images were acquired by widefield microscopy with an automated whole slide scanner. Nuclear versus cytoplasmic RBM20 localization was quantified manually in 3-4 slices per mouse heart for two mice per condition and a total of 250-500 cells.

### PicoSirius Red Staining

Hearts were processed for standard paraffin embedding. Sagittal sections were collected around the mid portion of each sample at 8 μm onto Superfrost Plus slides. Following deparaffinization and hydration with alcohols to water, the sections were stained with a solution of picrosirius red (0.5 g/500 ml saturated picric acid; Sigma) for 1 h at RT. Sections were then washed in two changes of acidified water (5 ml Glacial acetic acid/1 later distilled water), dehydrated in 100% ethanol and mounted in Permount. Images were acquired with an automated whole slide scanner.

### Echocardiography

Mice were anesthetized using 2-2.5% isoflurane (HDG9623V, Baxter Deutschland GmbH, Germany), while heart and respiratory rate were continually monitored. Cardiac echocardiography was performed using a Vevo 2100 Imaging System with a MS400 Transducer (both FUJIFILM VisualSonics, Inc., Canada), and B-mode and M-mode images of short axis and long axis were taken. During echocardiography, mice were placed on a heating pad to avoid a decrease in body temperature. Echocardiographic parameters were then analyzed using the VisualSonics VevoLab software.

### Vertical SDS Agarose Gel Electrophoresis (VAGE)

VAGE for detection of TTN protein isoforms was performed as described before⁶² with minor modifications. A piece of 5-10 mg heart tissue was lysed in 40 volumes (w/v) of VAGE sample buffer (8 M urea, 2 M thiourea, 3% SDS, 0.03% bromphenol blue, 0.05 M Tris-HCl, 75 mM DTT, pH 6.8) with a pistel in a microtube at 60° C. for 2-3 min. Subsequently, 50% glycerol buffer (50 ml H2O, 50 ml Ultrapure Glycerol, 1 Tablet protease inhibitor cocktail (11697498001, Roche)) was added (final concentration 12%) and the samples were processed for another 3-5 min at RT. After a cooling period of 5 min on ice, a centrifugation for 5 min at 16,000×g occurred. The supernatant was taken and stored at 80° C. The samples were thawed by heating to 60° C. for 2 min and analyzed using VAGE. After the gel run, the gel was fixed for 1 h in 50% methanol, 12% acetic acid 5% glycerol in ddH2O and dried overnight. The gel was rehydrated in H₂O, stained with Coomassie and scanned for quantification. Analysis and quantification was performed with the AIDA software.

### Whole-Genome Sequencing and Analysis

DNA for WGS was prepared by PCR-free library preparation according to the NEBNext Ultra II DNA PCR-free Library Prep kit (E7410L, NEB). Sequencing was performed by Illumina NextSeq 2000 P3 150PE.

Analysis of the sequencing data was performed using a customized Snakemake workflow. Raw FASTQ files were initially processed for 3′ adapter trimming from raw FASTQ files using cutadapt v.3.5⁶³. The trimmed reads were subsequently aligned by bwa-mem v.0.7.17⁶⁴ to a hybrid reference sequence of mouse genome mm10 concatenated with AAV vector backbone sequences comprising the N- and C-terminal components of SpRY-Cas9-ABE. The BAM files were sorted, marked for PCR duplicates and recalibrated for base quality scores using GATK v4.1.9.0. Three variant callers were applied for SNP (GATK Mutect2 v.4.1.9.0 (MU)⁶⁵ GATK HaplotypeCaller v.4.1.9.0 (HC)⁶⁵, Lofreq v.2.1.5 (LF)⁶⁶) and Indel (MU, HC, Scalpel v.0.5.4 (SC)⁶⁷) calling, respectively. For MU and HC, variant calling was performed in cohort mode using BAM files of all three tissue samples from the same individual. Therefore, allelic depth and frequency (AF) of reference and alternative alleles were recorded even for variants not present across all tissue types. For LF and SC, the default parameters were used to call variants from a single sample at a time. All variants were left-aligned and normalized using boftools (v.1.9)⁶⁸ to allow a comparison between variant callers. For further analysis, the allelic depth called by MU was used if present, and was otherwise replaced by values determined by HC. ANNOVAR (v.2020-06-08)⁶⁹ was used to add functional annotations to the detected variants. To identify variants with high confidence, we required a variant to (1) be called by at least two variant callers, (2) be covered by at least five reads per tissue type, and (3) have at least two alternative allele reads across all tissues. After this quality filtering step, tissue-overlapping variants were defined as variants present in all three tissues. To identify novel mutations with high confidence, variants overlapping with any known variant annotated by the mouse genome project⁷⁰ or dbSNP⁷¹ were excluded. From this pool of variants, tissue-specific variants were defined as variants with an AF>0 in the tissue in question and an AF=0 or non-measured variants in the other tissues. To further characterize tissue-specific variants, they were examined for a potential causation by the CRISPR base-editor treatment. For each variant, a section of +30 bases around its start site was investigated for sequence homology to the gRNA and PAM sequence. A custom script was developed for the sequence alignment and calculation of minimum edit distance, while only allowing 1 bp indels or mismatches in the seed region but not in the PAM site.

### RNA-Seg Variant Analysis

Analysis of the sequencing data was performed using a customized Snakemake workflow. Raw FASTQ files were aligned by STAR v2.7.9a in 2-pass mode⁵⁴ to the same hybrid reference sequence used in the WGS analysis. The BAM files were sorted and marked for PCR duplicates using GATK v4.1.9.064. Three variant callers were applied for variant calling: (GATK HaplotypeCaller v.4.1.9.0 (HC)⁶⁵, Strelka v.2.9.10 (ST)⁷² and Platypus v.0.8.1 (PL))⁷³. For HC, the sorted and marked reads were pre-processed as described in the GATK Best Practices for RNA-seq variant calling^{74,75}. For PL, sorted and marked reads were processed with Opossum v.0.2⁷⁶ and ST used sorted and marked reads as their input and was run in RNA mode. All algorithms called variants in cohort mode using BAM files of all three tissue samples from the same individual. All variants were normalized, left-aligned, and annotated as described in the WGS analysis section. For further analysis, the allelic depth called by PL was used if present, and was otherwise replaced by values determined by HC. To identify variants with high confidence, we required a variant to (1) be called by at least two out of three variant callers, (2) be covered by at least five reads (tissue were investigated individually), (3) have at least two alternative allele reads across all tissues, and 4) be located in exons, introns, or UTR3/5 regions. Known variants annotated by dbSNP or MGP were excluded, and the remaining variants were grouped into tissue-specific or tissue-overlapping variants.

For FIG. 12b, REDItools2⁷⁷ was utilized to extract all reads from the target region. Triplicate reads were summed up, and the fraction per base and position was calculated. Investigation of sequence similarity between gRNA regions surrounding the SNVs was performed as described in the WGS analysis.

Nuclei Isolation and snRNA-Seq

Nuclei from mouse hearts were isolated using previous protocols with some adaptations. Briefly, hearts were washed three times with PBS, minced and incubated with 5 ml 1× Red Blood Cell Lysis Buffer (Z3141, Promega) for 5 min with manual shaking. In total, 5 ml PBS was added, followed by centrifugation at 500×g/2 min and an additional washing step with 10 ml PBS. The pellet was resuspended in 1 ml homogenization buffer⁷⁸, and dounced 8× with pestle ‘A’ and 20× with pestle ‘B’ on ice. Nuclei were filtered through a 70 μM strainer, followed by a 40 μM strainer and a 20 μM strainer. Nuclei were centrifuged at 1000× g/5 min and pellet resuspended in 2 ml homogenization buffer. Nuclei solution was layered on 10 ml sucrose buffer⁷⁹ and centrifuged 1000× g/5 min. The pellet was washed with 2 ml homogenization buffer and resuspended in 0.2 ml PBS (calcium and magnesium free) with 2% BSA and 0.2 U/μl RNasin® Plus RNase inhibitor (N2615, Promega). Nuclei stained with Dapi were sorted by flow cytometry in FACS buffer. The gating strategy is depicted in the source data. Sequencing libraries were prepared with the Single Cell 5′ Reagent Kit v2 Dual Index (1000265, 10× Genomics) and sequencing was performed by NextSeq550 Mid 75 PE.

### SnRNA-Seg Analysis

SnRNA-seq data was aligned to the mouse reference mm10 (GENCODE vM23/Ensembl 98) using 10× Genomics' Cell Ranger 7.0. Downstream analysis on the gene count matrix was performed in R v4.2.1 and Seurat v4. At the pre-processing stage, the cells were filtered such that each cell has between 100 and 2500 active genes with non-zero counts. Cells exhibiting more than 1% counts belonging to mitochondrial genes were not included. The counts of each cell were log-normalized, and the 2000 most variable features were identified in each run separately. Pre-processed data from different runs were harmonized using the FindIntegration Anchors method in Seurat. Principal Component Analysis (PCA) was performed on the integrated data to identify the 30 largest contributors to gene expression profile variation. Clusters were identified using the Louvain algorithm⁸⁰ with resolution parameter 0.5. For visualization of the cell clusters, Uniform Manifold Approximation and Projection (UMAP) reduction⁸¹ was performed on the 30 PCs. Each cluster was mapped to a specific cell type using markers provided by the Heart Cell Atlas³⁷. Within each cluster, an additional PCA was performed on the pre-integration gene expression data to identify sub-cluster variation. UMAP visualization was obtained from the 5 largest PCs, while pairwise cell distances were calculated from the first two PCs using the Euclidean metric.

For cell type-specific comparison of inter-genotype variations, differential expression analysis was performed between the WT and P635L HOM cells within each cluster using the Wilcoxon Rank Sum test, with maximum P value 0.05. Per cell type, up to 15 up- and downregulated genes were identified. Their expression values were rescaled across cells such that their values were between 0 and 1, where 1 indicates maximum activation of the gene in a cell, and 0 indicates non-expression. An overall activation score was then calculated for each cell by averaging its rescaled expression values across the list of up-/downregulated genes. By scanning a threshold value between 0 and 1, the percentage of cells with activity scores above threshold was then used as a proxy for that population's expression of a set of genes. The critical threshold for comparison was chosen as the value where the genotype with downregulated activity drops below 50% active cells.

### Statistics & Reproducibility

GraphPad Prism software (v9.3.1) was used for statistical analysis except RNA-seq data which was analyzed in R. Data were analyzed with unpaired t tests, one-way or two-way ANOVA with Tukey's multiple comparison posttest or log-rank test. Name of the test, P value and number of biological replicates are indicated in each figure legend. Data are displayed as means±SEM. No statistical method was used to predetermine sample size. No data were excluded from the analyses. The experiments were not randomized, and the investigators were in most cases not blinded to allocation during experiments and outcome assessment.

### REFERENCES

1. Anzalone, A. V., Koblan, L. W. & Liu, D. R. Genome editing with CRISPR-Cas nucleases, base editors, transposases and prime editors. Nat. Biotechnol. 38, 824-844 (2020).
2. CRISPR Clinical Trials: A Update. Innovative Genomics Institute (IGI) https://innovativegenomics.org/news/crispr-clinical-trials-2022/(2022).
3. Verve Therapeutics, Inc. Open-label, Phase 1b, Single-ascending Dose and Optional re Dosing Study to Evaluate the Safety of VERVE-101 Administered to Patients With Heterozygous Familial Hypercholesterolemia, Atherosclerotic Cardiovascular Disease, and Uncontrolled Hypercholesterolemia. https://clinicaltrials.gov/ct2/show/NCT05398029 (2022)
4. Pérez-Palma, E., Gramm, M., Nürnberg, P., May, P. & Lal, D. Simple Clin Var: an interactive web server to explore and retrieve gene and disease variants aggregated in Clin Var database. Nucleic Acids Res. 47, W99-W105 (2019).
5. Long, C. et al. Prevention of muscular dystrophy in mice by CRISPR/Cas9-mediated editing of germline DNA. Science 345, 1184-1188 (2014).
6. Kaneko, M., Hashikami, K., Yamamoto, S., Matsumoto, H. & Nishimoto, T. Phospholamban ablation using CRISPR/Cas9 system improves mortality in a murine heart failure model. PLOS ONE 11, c0168486 (2016).
7. Ma, S. et al. Efficient correction of a hypertrophic cardiomyopathy mutation by ABEmax-NG. Circ. Res. 129, 895-908 (2021).
8. Ma, H. et al. Correction of a pathogenic gene mutation in human embryos. Nature 548, 413-419 (2017).
9. Pan, X. et al. In vivo Ryr2 editing corrects catecholaminergic polymorphic ventricular tachycardia. Circ. Res. 123, 953-963 (2018).
10. Amoasii, L. et al. Gene editing restores dystrophin expression in a canine model of Duchenne muscular dystrophy. Science 362, 86-91 (2018).
11. Moretti, A. et al. Somatic gene editing ameliorates skeletal and cardiac muscle failure in pig and human models of Duchenne muscular dystrophy. Nat. Med. 26, 207-214 (2020).
12. Rosenbaum, A. N., Agre, K. E. & Pereira, N. L. Genetics of dilated cardiomyopathy: practical implications for heart failure management. Nat. Rev. Cardiol. 17, 286-297 (2020).
13. XU, X.-R. et al. Fifteen-year mortality and prognostic factors in patients with dilated cardiomyopathy: persistent standardized application of drug therapy and strengthened management may bring about encouraging change in an aging society. J. Geriatr. Cardiol. 19, 335-342 (2022).
14. Verdonschot, J. A. J., Hazebroek, M. R., Ware, J. S., Prasad, S. K. & Heymans, S. R. B. Role of targeted therapy in dilated cardiomyopathy: the challenging road toward a personalized approach. J. Am. Heart Assoc. 8, e012514 (2019).
15. Refaat, M. M. et al. Genetic variation in the alternative splicing regulator RBM20 is associated with dilated cardiomyopathy. Heart Rhythm. 9, 390-396 (2012).
16. Koelemen, J., Gotthardt, M., Steinmetz, L. M. & Meder, B. RBM20-related cardiomyopathy: current understanding and future options. J. Clin. Med. 10, 4101 (2021).
17. Schneider, J. W. et al. Dysregulated ribonucleoprotein granules promote cardiomyopathy in RBM20 gene-edited pigs. Nat. Med. 26, 1788-1800 (2020).
18. Ihara, K. et al. A missense mutation in the RSRSP stretch of Rbm20 causes dilated cardiomyopathy and atrial fibrillation in mice. Sci. Rep. 10, 17894 (2020).
19. Fenix, A. M. et al. Gain-of-function cardiomyopathie mutations in RBM20 rewire splicing regulation and re-distribute ribonucleoprotein aggregates within processing bodies. Nat. Commun. 12, 6324 (2021).
20. Zaiss, A. K. & Muruve, D. A. Immune responses to adeno-associated virus vectors. Curr. Gene Ther. 5, 323-331 (2005).
21. Bijlani, S., Pang, K. M., Sivanandam, V., Singh, A. & Chatterjee, S. The role of recombinant AAV in precise genome editing. Front. Genome Editing 3, 799722 (2022).
22. Weinmann, J. et al. Identification of a myotropic AAV by massively parallel in vivo evaluation of barcoded capsid variants. Nat. Commun. 11, 5432 (2020).
23. Kingwell, K. Base editors hit the clinic. Nat. Rev. Drug Discov. 21, 545-547 (2022).
24. Briganti, F. et al. iPSC modeling of RBM20-deficient DCM identifies upregulation of RBM20 as a therapeutic strategy. Cell Rep. 32, 108117 (2020).
25. Zografos, T. A. & Katritsis, D. G. Natriuretic peptides as predictors of atrial fibrillation recurrences following electrical cardioversion. Arrhythm Electrophysiol. Rev. 2, 109-114 (2013).
26. Zhang, Y. et al. RBM20 phosphorylation and its role in nucleocytoplasmic transport and cardiac pathogenesis. FASEB J. 36, e22302 (2022).
27. Wang, C. et al. RBM20S639G mutation is a high genetic risk factor for premature death through RNA-protein condensates. J. Mol. Cell Cardiol. 165, 115-129 (2022).
28. Nishiyama, T. et al. Precise genomic editing of pathogenic mutations in RBM20 rescues dilated cardiomyopathy. Sci. Transl. Med. 14, eade 1633 (2022).
29. Zi, M. et al. Cardiac hypertrophy or failure? A systematic evaluation of the transverse aortic constriction model in C57BL/6NTac and C57BL/6J substrains. Curr. Res. Physiol. 1, 1-10 (2019).
30. Walton, R. T., Christie, K. A., Whittaker, M. N. & Kleinstiver, B. P. Unconstrained genome targeting with near-PAMless engineered CRISPR-Cas9 variants. Science 368, 290-296 (2020).
31. Miller, S. M. et al. Continuous evolution of SpCas9 variants compatible with non-G PAMs. Nat. Biotechnol. 38, 471-481 (2020).
32. Huang, T. P. et al. Circularly permuted and PAM-modified Cas9 variants broaden the targeting scope of base editors. Nat. Biotechnol. 37, 626-631 (2019).
33. Zhu, C. et al. Single-molecule, full-length transcript isoform sequencing reveals disease-associated RNA isoforms in cardiomyocytes. Nat. Commun. 12, 4203 (2021).
34. Levy, J. M. et al. Cytosine and adenine base editing of the brain, liver, retina, heart and skeletal muscle of mice via adeno-associated viruses. Nat. Biomed. Eng. 4, 97-110 (2020).
35. Richter, M. F. et al. Phage-assisted evolution of an adenine base editor with improved Cas domain compatibility and activity. Nat. Biotechnol. 38, 883-891 (2020).
36. Zhou, P. & Pu, W. T. Recounting cardiac cellular composition. Circ. Res. 118, 368-370 (2016).
37. Litvinuková, M. et al. Cells of the adult human heart. Nature 588, 466-472 (2020).
38. Guo, W. et al. RBM20, a gene for hereditary cardiomyopathy, regulates titin splicing. Nat. Med. 18, 766-773 (2012),
39. Zuo, E. et al. GOTI, a method to identify genome-wide off-target effects of genome editing in mouse embryos. Nat. Protoc. 15, 3009-3029 (2020).
40. Grünewald, J. et al. Transcriptome-wide off-target RNA editing induced by CRISPR-guided DNA base editors. Nature 569, 433-437 (2019).
41. Rees, H. A., Wilson, C., Doman, J. L. & Liu, D. R. Analysis and minimization of cellular RNA editing by DNA adenine base editors. Sci. Adv. 5, caax5717 (2019).
42. Reichart, D. et al. Efficient in vivo genome editing prevents hypertrophic cardiomyopathy in mice. Nat. Med. 29, 412-421 (2023).
43. Ertl, H. C. J. Immunogenicity and toxicity of AAV gene therapy. Front. Immunol. 13, 975803 (2022)
44. Au, H. K. E., Isalan, M. & Mielcarek, M. Gene therapy advances: a meta-analysis of AAV usage in clinical settings. Front. Med. 8, 809118 (2022).
45. Morales, A. & Hershberger, R. E. The rationale and timing of molecular genetic testing for dilated cardiomyopathy. Can. J. Cardiol. 31, 1309-1312 (2015).
46. Zuo, E. et al. Cytosine base editor generates substantial off-target single-nucleotide variants in mouse embryos. Science 364, 289-292 (2019).
47. Rothgangl, T. et al. In vivo adenine base editing of PCSK9 in macaques reduces LDL cholesterol levels. Nat. Biotechnol. 1-9 https://doi.org/10.1038/s41587-021-00933-4 (2021).
48. Reichart, D. et al. Pathogenic variants damage cell composition and single cell transcription in cardiomyopathies. Science 377, eabo1984 (2022).
49. Koblan, L. W. et al. In vivo base editing rescues Hutchinson-Gilford progeria syndrome in mice. Nature 589, 608-614 (2021).
50. Haar, J., Krämer, C. & Grimm, D. Lab-scale production of recombinant adeno-associated viruses (AAV) for expression of optogenetic elements. Methods Mol. Biol. 2173, 83-100 (2020).
51. Clement, K. et al. CRISPResso2 provides accurate and rapid genome editing sequence analysis. Nat. Biotechnol. 37, 224-226 (2019).
52. Picelli, S. et al. Smart-seq2 for sensitive full-length transcriptome profiling in single cells. Nat. Methods 10, 1096-1098 (2013).
53. Mölder, F. et al. Sustainable data analysis with Snakemake. F1000Res 10, 33 (2021).
54. Dobin, A. et al. STAR: ultrafast universal RNA-seq aligner, Bioinformatics 29, 15-21 (2013).
55. Love, M. L., Huber, W. & Anders, S. Moderated estimation of fold change and dispersion for RNA-seq data with DESeq2. Genome Biol. 15, 550 (2014).
56. R Core Team. R: A language and environment for statistical computing. R Foundation for Statistical Computing https://www.r-project.org/(Vienna, Austria, 2022).
57. Liao, Y., Smyth, G. K. & Shi, W. The R package Rsubread is easier, faster, cheaper and better for alignment and quantification of RNA sequencing reads. Nucleic Acids Res. 47, e47 (2019).
58. Zhou, Y. et al. Metascape provides a biologist-oriented resource for the analysis of systems-level datasets. Nat. Commun. 10, 1523 (2019).
59. Shen, S. et al. rMATS: robust and flexible detection of differential alternative splicing from replicate RNA-Seq data. Proc. Natl Acad. Sci. USA 111, E5593-E5601 (2014).
60. Ackers-Johnson, M. et al. A simplified, langendorff-free method for concomitant isolation of viable cardiac myocytes and nonmyocytes from the adult mouse heart. Circ. Res. 119, 909-920 (2016).
61. Klickstein, J. A., Mukkavalli, S. & Raman, M. AggreCount: an unbiased image analysis tool for identifying and quantifying cellular aggregates in a spatially defined manner. J. Biol. Chem. 295, 17672-17683 (2020).
62. Warren, C. M., Krzesinski, P. R. & Greaser, M. L. Vertical agarose gel electrophoresis and electroblotting of high-molecular-weight proteins. Electrophoresis 24, 1695-1702 (2003).
63. Martin, M. Cutadapt removes adapter sequences from high-throughput sequencing reads. EMBnet.journal 17, 10-12 (2011).
64. Li, H. & Durbin, R. Fast and accurate short read alignment with Burrows-Wheeler transform. Bioinformatics 25, 1754-1760 (2009).
65. Cibulskis, K. et al. Sensitive detection of somatic point mutations in impure and heterogeneous cancer samples. Nat. Biotechnol. 31, 213-219 (2013).
66. Wilm, A. et al. LoFreq: a sequence-quality aware, ultra-sensitive variant caller for uncovering cell-population heterogeneity from high-throughput sequencing datasets. Nucleic Acids Res. 40, 11189-11201 (2012).
67. Fang, H. et al. Indel variant analysis of short-read sequencing data with Scalpel. Nat. Protoc. 11, 2529-2548 (2016).
68. Danecek, P. et al. Twelve years of SAMtools and BCFtools. Gigascience 10, giab008 (2021).
69. Wang, K., Li, M. & Hakonarson, H. ANNOVAR: functional annotation of genetic variants from high-throughput sequencing data. Nucleic Acids Res. 38, e164 (2010).
70. Keane, T. M. et al. Mouse genomic variation and its effect on phenotypes and gene regulation. Nature 477, 289-294 (2011).
71. Sherry, S. T. et al. dbSNP: the NCBI database of genetic variation. Nucleic Acids Res. 29, 308-311 (2001).
72. Kim, S. et al. Strelka2: fast and accurate calling of germline and somatic variants. Nat. Methods 15, 591-594 (2018).
73. Rimmer, A. et al. Integrating mapping-, assembly- and haplotype-based approaches for calling variants in clinical sequencing applications. Nat. Gener. 46, 912-918 (2014).
74. Mckenna, A. et al. The Genome Analysis Toolkit: a MapReduce framework for analyzing next-generation DNA sequencing data. Genome Res. 20, 1297-1303 (2010).
75. DePristo, M. A. et al. A framework for variation discovery and genotyping using next-generation DNA sequencing data. Nat. Genet. 43, 491-498 (2011).
76. Oikkonen, L. & Lise, S. Making the most of RNA-seq: pre-processing sequencing data with Opossum for reliable SNP variant detection. Wellcome Open Res. 2, 6 (2017).
77. Picardi, E. & Pesole, G. REDItools: high-throughput RNA editing detection made easy. Bioinformatics 29, 1813-1814 (2013).
78. Krishnaswami, S. R. et al. Using single nuclei for RNA-seq to capture the transcriptome of postmortem neurons. Nat. Protoc. 11, 499-524 (2016).
79. Cui, M. et al. Dynamic Transcriptional Responses to Injury of Regenerative and Non-regenerative Cardiomyocytes Revealed by Single-Nucleus RNA Sequencing. Dev Cell 53, 102-116.e8 (2020).
80. Cui, M. & Olson, E. N. Protocol for single-nucleus transcriptomics of diploid and tetraploid cardiomyocytes in murine hearts. STAR Protoc. 1, 100049 (2020).
81. Blondel, V. D., Guillaume, J.-L., Lambiotte, R. & Lefebvre, E. Fast unfolding of communities in large networks. J. Stat. Mech. 2008, P10008 (2008).
82. McInnes, L., Healy, J., Saul, N. & Großberger, L. UMAP: Uniform Manifold Approximation and Projection. Journal of Open Source Software 3, 861 (2018).
83. Lennermann, D. C. et al. Deep phenotyping of two preclinical mouse models and a cohort of RBM20 mutation carriers reveals no sex-dependent disease severity in RBM20 cardiomyopathy. Am J Physiol Heart Circ Physiol 323, H1296-H1310 (2022).

### Exemplary Embodiments

Exemplary embodiments provided in accordance with the presently disclosed subject matter include, but are not limited to, the claims and the following embodiments:
Embodiment 1. A method for correcting a point mutation at the RBM20 locus in a cell, the method comprising: introducing into the cell (i) a single guide RNA (sgRNA) targeting a sequence comprising the point mutation and (ii) a base editor (BE), wherein the sgRNA binds to the base editor and directs it to the target sequence, whereupon the base editor corrects the point mutation at the RBM20 locus in the cell.
Embodiment 2. The method of embodiment 1, wherein the method further comprises isolating the cell from the subject prior to the introducing of the sgRNA and the BE.
Embodiment 3. The method of embodiment 1 or 2, wherein the point mutation at the RBM20 locus comprises a substitution at an amino acid position comprising 83, 455, 535, 633, 634, 635, 636, 637, 638, 703, 716, 783, 831, 888, 913, 914, 1031, 1081, 1182, 1206, or a combination thereof; and wherein the substitutions and the positions are determined with reference to SEQ ID NO: 10.
Embodiment 4. The method of embodiment 3, wherein the point mutation at the RBM20 locus comprises a substitution at an amino acid position comprising 633, 634, 635, 636, 637, 638, or a combination thereof.
Embodiment 5. The method of embodiment 4, wherein the point mutation at the RBM20 locus comprises a substitution at amino acid position 633, 634, or a combination thereof.
Embodiment 6. The method of embodiment 5, wherein the substitution comprises P633L, R634Q, or a combination thereof.
Embodiment 7. The method of any one of embodiments 1 to 6, wherein the sgRNA comprises a sequence having about 80% or greater identity to any one of SEQ ID NOs: 1-9.
Embodiment 8. The method of any one of embodiments 1 to 7, wherein the sgRNA comprises a sequence of any one of SEQ ID NOs: 1-9.
Embodiment 9. The method of any one of embodiments 1 to 8, wherein the BE is an adenine base editor (ABE) or a cytidine base editor (CBE).
Embodiment 10. The method of any one of embodiments 1 to 9, wherein the BE comprises an RNA-guided catalytically impaired nuclease fused to a nucleobase deaminase enzyme.
Embodiment 11. The method of embodiment 10, wherein the RNA-guided catalytically impaired nuclease is a dead Cas9 (dCas9), dCas12, or Cas9 nickase (Cas9n), or a derivative thereof.
Embodiment 12. The method of embodiment 10 or 11, wherein the RNA-guided catalytically impaired nuclease is an engineered Cas9n.
Embodiment 13. The method of embodiment 12, wherein the engineered Cason is Cas9n-NRNH, Cas9n-NRTH, Cas9n-NRCH, CP-1041, or SpRY.
Embodiment 14. The method of embodiment 10, wherein the nucleobase deaminase enzyme is a single-stranded DNA (ssDNA)-specific deaminase enzyme.
Embodiment 15. The method of embodiment 14, wherein the deaminase enzyme is an adenine deaminase or a cytidine deaminase.
Embodiment 16. The method of any one of embodiments 1 to 15, wherein the BE is BE1, BE2, BE3, BE4, ABE6.3, ABE7.8, ABE7.9, ABE7.10, BE4max, AncBE4max, ABEmax, ABE8c, ABE-SpRY, CBE-SpRY, ABE-CP-1041, and CBE-CP-1041.
Embodiment 17. The method of any one of embodiments 1 to 16, wherein the sgRNA and the BE are introduced into the cell in one or more expression cassettes.
Embodiment 18. The method of embodiment 17, wherein the BE is present in one expression cassette.
Embodiment 19. The method of embodiment 17, wherein the BE is present in two expression cassettes, wherein an active BE is packaged in the cell through intein-mediated trans-splicing.
Embodiment 20. The method of any one of embodiments 17 to 19, wherein the expression cassette comprises a promoter.
Embodiment 21. The method of embodiment 20, wherein the promoter is a CAG promoter.
Embodiment 22. The method of embodiment 20, wherein the promoter is a human cardiac troponin T (hTNNT2) promoter.
Embodiment 23. The method of any one of embodiments 1 to 22, wherein the sgRNA and the BE are introduced into the cell using a recombinant adeno-associated virus (rAAV) vector.
Embodiment 24. The method of embodiment 23, wherein the rAAV vector is an AAVMYO vector.
Embodiment 25. The method of any one of embodiments 1 to 22, wherein the sgRNA and the BE are introduced into the cell as a ribonucleoprotein (RNP).
Embodiment 26. The method of embodiment 27, wherein the RNP is introduced into the cell by electroporation.
Embodiment 27. The method of any one of embodiments 1 to 26, wherein the cell is an induced pluripotent stem cell (iPSC) or a cardiomyocyte derived from the iPSC (CM-iPSC).
Embodiment 28. A method for treating or preventing a subject having dilated cardiomyopathy (DCM), comprising (i) genetically modifying a cell from the subject using the method of any one of embodiments 1 to 27, and (ii) reintroducing the cell into the subject, wherein the reintroducing is effective to treat or prevent the subject having DCM.
Embodiment 29. The method of embodiment 28, wherein the subject has a point mutation at the RBM20 locus.
Embodiment 30. The method of embodiment 29, wherein the point mutation at the RBM20 locus comprises a substitution at an amino acid position comprising 83, 455, 535, 633, 634, 635, 636, 637, 638, 703, 716, 783, 831, 888, 913, 914, 1031, 1081, 1182, 1206, or a combination thereof; and wherein the substitutions and the positions are determined with reference to SEQ ID NO: 10.
Embodiment 31. The method of embodiment 29 or 30, wherein the point mutation at the RBM20 locus comprises a substitution at an amino acid position comprising 633, 634, 635, 636, 637, 638, or a combination thereof.
Embodiment 32. The method of embodiment 31, wherein the point mutation at the RBM20 locus comprises a substitution at amino acid position 633, 634, or a combination thereof.
Embodiment 33. The method of embodiment 32, wherein the substitution comprises P633L, R634Q, or a combination thereof.
Embodiment 34. The method of any one of embodiments 28 to 33, wherein the cell is reintroduced into the subject by systemic delivery.
Embodiment 35. The method of any one of embodiments 28 to 33, wherein the cell is reintroduced into the subject by local delivery.
Embodiment 36. The method of embodiment 35, wherein the local delivery is intrafemoral or intrahepatic.
Embodiment 37. The method of any one of embodiments 28 to 36, wherein the cell is cultured, expanded, selected, and/or induced to undergo differentiation in vitro prior to being reintroduced into the subject.
Embodiment 38. An sgRNA that specifically targets the RBM20 gene comprising a sequence having about 80% or greater identity to any one of SEQ ID NOs: 1-9.
Embodiment 39. An iPSC comprising the sgRNA of embodiment 38 and a base editor (BE) comprising an RNA-guided catalytically impaired nuclease fused to a nucleobase deaminase enzyme.
Embodiment 40. A cardiomyocyte derived from the iPSC of embodiment 39.
Embodiment 41. A pharmaceutical composition comprising a plurality of iPSCs of embodiment 39, or a plurality of cardiomyocytes of embodiment 40.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

INFORMAL SEQUENCE LISTING

| SEQ ID NO: | Name | Sequence |
|---|---|---|
| SEQ ID NO: 1 | P633L_gRNA_1 | ACCGCAGCCTTTCTGGGCCA |
| SEQ ID NO: 2 | P633L_gRNA_2 | AGACCGCAGCCTTTCTGGGC |
| SEQ ID NO: 3 | P633L_gRNA_3 | TACGAGACCGCAGCCTTTCT |
| SEQ ID NO: 4 | P633L_gRNA_4 | CTACGAGACCGCAGCCTTTC |
| SEQ ID NO: 5 | R634Q_gRNA_1 | GAGGCCGCAGTCTCGTAGTCC |
| SEQ ID NO: 6 | R634Q_gRNA_2 | GGCCGCAGTCTOGTAGTCCG |
| SEQ ID NO: 7 | R634Q_gRNA_3 | GCCGCAGTCTCGTAGTCCGG |
| SEQ ID NO: 8 | R634Q_gRNA_4 | CCGCAGTCTCGTAGTCCGGT |
| SEQ ID NO: 9 | R634Q_gRNA_5 | CGCAGTCTCGTAGTCCGGTG |

SEQ ID NO: 10 WT human RBM20 amino acid sequence
MVLAAAMSQDADPSGPEQPDRVACSVPGARASPAPSGPRGMQQPPPPPQPPP
PPQAGLPQIIQNAAKLLDKNPFSVSNPNPLLPSPASLQLAQLQAQLTLHRLKLAQTAV
TNNTAAATVLNQVLSKVAMSQPLFNQLRHPSVITGPHGHAGVPQHAAAIPSTRFPSN
AIAFSPPSQTRGPGPSMNLPNQPPSAMVMHPFTGVMPQTPGQPAVILGIGKTGPAPAT
AGFYEYGKASSGQTYGPETDGQPGFLPSSASTSGSVTYEGHYSHTGQDGQAAFSKDF
YGPNSQGSHVASGFPAEQAGGLKSEVGPLLQGTNSQWESPHGFSGQSKPDLTAGPM
WPPPHNQPYELYDPEEPTSDRTPPSFGGRLNNSKQGFIGAGRRAKEDQALLSVRPLQ
AHELNDFHGVAPLHLPHICSICDKKVFDLKDWELHVKGKLHAQKCLVFSENAGIRCI
LGSAEGTLCASPNSTAVYNPAGNEDYASNLGTSYVPIPARSFTQSSPTFPLASVGTTF
AQRKGAGRVVHICNLPEGSCTENDVINLGLPFGKVTNYILMKSTNQAFLEMAYTEA
AQAMVQYYQEKSAVINGEKLLIRMSKRYKELQLKKPGKAVAAIIQDIHSQRERDMF
READRYGPERPRSRSPVSRSLSPRSHTPSFTSCSSSHSPPGPSRADWGNGRDSWEHSP
YARREEERDPAPWRDNGDDKRDRMDPWAHDRKHHPRQLDKAELDERPEGGRPHR
EKYPRSGSPNLPHSVSSYKSREDGYYRKEPKAKSDKYLKQQQDAPGRSRRKDEARL
RESRHPHPDDSGKEDGLGPKVTRAPEGAKAKQNEKNKTKRTDRDQEGADDRKENT
MAENEAGKEEQEGMEESPQSVGRQEKEAEFSDPENTRTKKEQDWESESEAEGESWY
PTNMEELVTVDEVGEEEDFIVEPDIPELEEIVPIDQKDKICPETCLCVTTTLDLDLAQD
FPKEGVKAVGNGAAEISLKSPRELPSASTSCPSDMDVEMPGLNLDAERKPAESETGLS
LEDSDCYEKEAKGVESSDVHPAPTVQQMSSPKPAEERARQPSPFVDDCKTRGTPEDG
ACEGSPLEEKASPPIETDLQNQACQEVLTPENSRYVEMKSLEVRSPEYTEVELKQPLS
LPSWEPEDVFSELSIPLGVEFVVPRTGFYCKLQGLFYTSEETAKMSHCRSAVHYRNLQ
KYLSQLAEEGLKETEGADSPRPEDSGIVPRFERKKL

What is claimed is:

1. A method for correcting a point mutation at the RBM20 locus in a cell, the method comprising:
introducing into the cell (i) a single guide RNA (sgRNA) targeting a sequence comprising the point mutation and (ii) a base editor (BE),
wherein the sgRNA binds to the base editor and directs it to the target sequence, whereupon the base editor corrects the point mutation at the RBM20 locus in the cell.

2. The method of claim 1, wherein the method further comprises isolating the cell from the subject prior to the introducing of the sgRNA and the BE.

3. The method of claim 1 or 2, wherein the point mutation at the RBM20 locus comprises a substitution at an amino acid position comprising 83, 455, 535, 633, 634, 635, 636, 637, 638, 703, 716, 783, 831, 888, 913, 914, 1031, 1081, 1182, 1206, or a combination thereof; and wherein the substitutions and the positions are determined with reference to SEQ ID NO: 10.

4. The method of claim 3, wherein the point mutation at the RBM20 locus comprises a substitution at an amino acid position comprising 633, 634, 635, 636, 637, 638, or a combination thereof.

5. The method of claim 4, wherein the point mutation at the RBM20 locus comprises a substitution at amino acid position 633, 634, or a combination thereof.

6. The method of claim 5, wherein the substitution comprises P633L, R634Q, or a combination thereof.

7. The method of claim 1, wherein the sgRNA comprises a sequence having about 80% or greater identity to any one of SEQ ID NOs: 1-9.

8. The method of claim 1, wherein the sgRNA comprises a sequence of any one of SEQ ID NOs: 1-9.

9. The method of claim 1, wherein the BE is an adenine base editor (ABE) or a cytidine base editor (CBE).

10. The method of claim 1, wherein the BE comprises an RNA-guided catalytically impaired nuclease fused to a nucleobase deaminase enzyme.

11. The method of claim 10, wherein the RNA-guided catalytically impaired nuclease is a dead Cas9 (dCas9), dCas12, or Cas9 nickase (Cas9n), or a derivative thereof.

12. The method of claim 10, wherein the RNA-guided catalytically impaired nuclease is an engineered Cas9n.

13. The method of claim 12, wherein the engineered Cas9n is Cason-NRNH, Cas9n-NRTH, Cas9n-NRCH, CP-1041, or SpRY.

14. The method of claim 10, wherein the nucleobase deaminase enzyme is a single-stranded DNA (ssDNA)-specific deaminase enzyme.

15. The method of claim 14, wherein the deaminase enzyme is an adenine deaminase or a cytidine deaminase.

16. The method of claim 1, wherein the BE is BE1, BE2, BE3, BE4, ABE6.3, ABE7.8, ABE7.9, ABE7.10, BE4max, AncBE4max, ABEmax, ABE8e, ABE-SpRY, CBE-SpRY, ABE-CP-1041, and CBE-CP-1041.

17. The method of claim 1, wherein the sgRNA and the BE are introduced into the cell in one or more expression cassettes.

18. The method of claim 17, wherein the BE is present in one expression cassette.

19. The method of claim 17, wherein the BE is present in two expression cassettes, wherein an active BE is packaged in the cell through intein-mediated trans-splicing.

20. The method of claim 17, wherein the expression cassette comprises a promoter.

21. The method of claim 20, wherein the promoter is a CAG promoter.

22. The method of claim 20, wherein the promoter is a human cardiac troponin T (hTNNT2) promoter.

23. The method of claim 1, wherein the sgRNA and the BE are introduced into the cell using a recombinant adeno-associated virus (rAAV) vector.

24. The method of claim 23, wherein the rAAV vector is an AAVMYO vector.

25. The method of claim 1, wherein the sgRNA and the BE are introduced into the cell as a ribonucleoprotein (RNP).

26. The method of claim 27, wherein the RNP is introduced into the cell by electroporation.

27. The method of claim 1, wherein the cell is an induced pluripotent stem cell (iPSC) or a cardiomyocyte derived from the iPSC (CM-iPSC).

28. A method for treating or preventing a subject having dilated cardiomyopathy (DCM), comprising (i) genetically modifying a cell from the subject using the method of claim 1, and (ii) reintroducing the cell into the subject, wherein the reintroducing is effective to treat or prevent the subject having DCM.

29. The method of claim 28, wherein the subject has a point mutation at the RBM20 locus.

30. The method of claim 29, wherein the point mutation at the RBM20 locus comprises a substitution at an amino acid position comprising 83, 455, 535, 633, 634, 635, 636, 637, 638, 703, 716, 783, 831, 888, 913, 914, 1031, 1081, 1182, 1206, or a combination thereof; and wherein the substitutions and the positions are determined with reference to SEQ ID NO: 10.

31. The method of claim 29 or 30, wherein the point mutation at the RBM20 locus comprises a substitution at an amino acid position comprising 633, 634, 635, 636, 637, 638, or a combination thereof.

32. The method of claim 31, wherein the point mutation at the RBM20 locus comprises a substitution at amino acid position 633, 634, or a combination thereof.

33. The method of claim 32, wherein the substitution comprises P633L, R634Q, or a combination thereof.

34. The method of claim 28, wherein the cell is reintroduced into the subject by systemic delivery.

35. The method of claim 28, wherein the cell is reintroduced into the subject by local delivery.

36. The method of claim 35, wherein the local delivery is intrafemoral or intrahepatic.

37. The method of claim 28, wherein the cell is cultured, expanded, selected, and/or induced to undergo differentiation in vitro prior to being reintroduced into the subject.

38. An sgRNA that specifically targets the RBM20 gene comprising a sequence having about 80% or greater identity to any one of SEQ ID NOs: 1-9.

39. An iPSC comprising the sgRNA of claim 38 and a base editor (BE) comprising an RNA-guided catalytically impaired nuclease fused to a nucleobase deaminase enzyme.

40. A cardiomyocyte derived from the iPSC of claim 39.

41. A pharmaceutical composition comprising a plurality of iPSCs of claim 39, or a plurality of cardiomyocytes of claim 40.


SEQUENCE LISTING (22 sequences)

SEQ ID NO: 1 - RNA, 20 nt, synthetic construct
  source 1..20: mol_type = other RNA
  ncRNA 1..20: ncRNA_class = guide_RNA
accgcagcctttctgggcca

SEQ ID NO: 2 - RNA, 20 nt, synthetic construct
  source 1..20: mol_type = other RNA
  ncRNA 1..20: ncRNA_class = guide_RNA
agaccgcagcctttctgggc

SEQ ID NO: 3 - RNA, 20 nt, synthetic construct
  source 1..20: mol_type = other RNA
  ncRNA 1..20: ncRNA_class = guide_RNA
tacgagaccgcagcctttct

SEQ ID NO: 4 - RNA, 20 nt, synthetic construct
  source 1..20: mol_type = other RNA
  ncRNA 1..20: ncRNA_class = guide_RNA
ctacgagaccgcagcctttc

SEQ ID NO: 5 - RNA, 21 nt, synthetic construct
  source 1..21: mol_type = other RNA
  ncRNA 1..21: ncRNA_class = guide_RNA
gaggccgcagtctcgtagtcc

SEQ ID NO: 6 - RNA, 20 nt, synthetic construct
  source 1..20: mol_type = other RNA
  ncRNA 1..20: ncRNA_class = guide_RNA
ggccgcagtctcgtagtccg

SEQ ID NO: 7 - RNA, 20 nt, synthetic construct
  source 1..20: mol_type = other RNA
  ncRNA 1..20: ncRNA_class = guide_RNA
gccgcagtctcgtagtccgg

SEQ ID NO: 8 - RNA, 20 nt, synthetic construct
  source 1..20: mol_type = other RNA
  ncRNA 1..20: ncRNA_class = guide_RNA
ccgcagtctcgtagtccggt

SEQ ID NO: 9 - RNA, 20 nt, synthetic construct
  source 1..20: mol_type = other RNA
  ncRNA 1..20: ncRNA_class = guide_RNA
cgcagtctcgtagtccggtg

SEQ ID NO: 10 - protein, 1227 aa, synthetic construct
MVLAAAMSQDADPSGPEQPDRVACSVPGARASPAPSGPRGMQQPPPPPQPPPPPQAGLPQIIQNAAKLLDKNPFSVSNPNPLLPSPASLQLAQLQAQLTLHRLKLAQTAVTNNTAAATVLNQVLSKVAMSQPLFNQLRHPSVITGPHGHAGVPQHAAAIPSTRFPSNAIAFSPPSQTRGPGPSMNLPNQPPSAMVMHPFTGVMPQTPGQPAVILGIGKTGPAPATAGFYEYGKASSGQTYGPETDGQPGFLPSSASTSGSVTYEGHYSHTGQDGQAAFSKDFYGPNSQGSHVASGFPAEQAGGLKSEVGPLLQGTNSQWESPHGFSGQSKPDLTAGPMWPPPHNQPYELYDPEEPTSDRTPPSFGGRLNNSKQGFIGAGRRAKEDQALLSVRPLQAHELNDFHGVAPLHLPHICSICDKKVFDLKDWELHVKGKLHAQKCLVFSENAGIRCILGSAEGTLCASPNSTAVYNPAGNEDYASNLGTSYVPIPARSFTQSSPTFPLASVGTTFAQRKGAGRVVHICNLPEGSCTENDVINLGLPFGKVTNYILMKSTNQAFLEMAYTEAAQAMVQYYQEKSAVINGEKLLIRMSKRYKELQLKKPGKAVAAIIQDIHSQRERDMFREADRYGPERPRSRSPVSRSLSPRSHTPSFTSCSSSHSPPGPSRADWGNGRDSWEHSPYARREEERDPAPWRDNGDDKRDRMDPWAHDRKHHPRQLDKAELDERPEGGRPHREKYPRSGSPNLPHSVSSYKSREDGYYRKEPKAKSDKYLKQQQDAPGRSRRKDEARLRESRHPHPDDSGKEDGLGPKVTRAPEGAKAKQNEKNKTKRTDRDQEGADDRKENTMAENEAGKEEQEGMEESPQSVGRQEKEAEFSDPENTRTKKEQDWESESEAEGESWYPTNMEELVTVDEVGEEEDFIVEPDIPELEEIVPIDQKDKICPETCLCVTTTLDLDLAQDFPKEGVKAVGNGAAEISLKSPRELPSASTSCPSDMDVEMPGLNLDAERKPAESETGLSLEDSDCYEKEAKGVESSDVHPAPTVQQMSSPKPAEERARQPSPFVDDCKTRGTPEDGACEGSPLEEKASPPIETDLQNQACQEVLTPENSRYVEMKSLEVRSPEYTEVELKQPLSLPSWEPEDVFSELSIPLGVEFVVPRTGFYCKLCGLFYTSEETAKMSHCRSAVHYRNLQKYLSQLAEEGLKETEGADSPRPEDSGIVPRFERKKL

SEQ ID NO: 11 - protein, 6 aa, synthetic construct
PRSRSP

SEQ ID NO: 12 - DNA, 17 bp, synthetic construct
  source 1..17: mol_type = other DNA
cggccaagttctcgaag

SEQ ID NO: 13 - DNA, 17 bp, synthetic construct
  source 1..17: mol_type = other DNA
cggctaagttctcgaag

SEQ ID NO: 14 - DNA, 17 bp, synthetic construct
  source 1..17: mol_type = other DNA
cggccaggttctcgaag

SEQ ID NO: 15 - DNA, 25 bp, synthetic construct
  source 1..25: mol_type = other DNA
gtatggtccagagcggctacgttct

SEQ ID NO: 16 - RNA, 25 nt, synthetic construct
  source 1..25: mol_type = other RNA
  ncRNA 1..25: ncRNA_class = guide_RNA
gtatggtccagagcggctacgttct

SEQ ID NO: 17 - RNA, 25 nt, synthetic construct
  source 1..25: mol_type = other RNA
  ncRNA 1..25: ncRNA_class = guide_RNA
gtatggtccagagcggccacgctct

SEQ ID NO: 18 - RNA, 25 nt, synthetic construct
  source 1..25: mol_type = other RNA
  ncRNA 1..25: ncRNA_class = guide_RNA
gtatggcccagagcggccacgttct

SEQ ID NO: 19 - RNA, 25 nt, synthetic construct
  source 1..25: mol_type = other RNA
  ncRNA 1..25: ncRNA_class = guide_RNA
gtatggtccagagcggccacgtcct

SEQ ID NO: 20 - RNA, 25 nt, synthetic construct
  source 1..25: mol_type = other RNA
  ncRNA 1..25: ncRNA_class = guide_RNA
gtatggtccagagcggccacgttcc

SEQ ID NO: 21 - RNA, 21 nt, synthetic construct
  source 1..21: mol_type = other RNA
  ncRNA 1..21: ncRNA_class = guide_RNA
tggtccagagcggccacgttc

SEQ ID NO: 22 - RNA, 21 nt, synthetic construct
  source 1..21: mol_type = other RNA
ccacggtctcgaagtccaatg